(19) United States
(12) Patent Application Publication
(10) Pub. No.: US 2016/0208339 A1
(43) Pub. Date: Jul. 21, 2016

(54) BIOMARKERS CORRELATED TO PARP INHIBITOR TREATMENT SUCCESS IN AML PATIENTS

(71) Applicant: KING'S COLLEGE LONDON, London (GB)

(72) Inventors: Chi Wai Eric So, London (GB); Maria Teresa Esposito, London (GB)

(21) Appl. No.: 15/022,637

(22) PCT Filed: Sep. 17, 2014
(86) PCT No.: PCT/GB2014/052810

§ 371 Date: Mar. 17, 2016

(30) Foreign Application Priority Data
Sep. 17, 2013 (GB) 1316526.1

(51) Int. Cl. C12Q 1/68; A61K 31/4184; A61K 45/06; A61K 31/502

(52) U.S. Cl. CPC C12Q 1/6886; A61K 31/502; A61K 31/4184; A61K 45/06; C12Q 2600/106

(57) ABSTRACT

In one aspect, there is provided a method for predicting responsiveness of a subject to a poly-(ADP-ribose)-polymerase (PARP) inhibitor for treating acute myeloid leukaemia (AML), the method comprising determining whether a chromosomal abnormality selected from t(8;21), t(15;17), t(16;16) and inv(16) is present in a sample obtained from the subject; wherein the presence of the chromosomal abnormality is indicative of responsiveness of the subject to the PARP inhibitor for treating AML.

### FIELD OF THE INVENTION

The present invention relates generally to the field of cancer diagnosis and treatment. In particular, the invention relates to methods for determining whether subjects will respond to an anti-cancer therapy. The invention is particularly applicable for identifying patients who will respond to administration of a poly-(ADP-ribose)-polymerase (PARP) inhibitor in the treatment of acute myeloid leukaemia (AML). The invention also relates to improved methods for treating AML involving selective administration of PARP inhibitors to responsive subjects.

### BACKGROUND

Acute myeloid leukaemia (AML) is a group of heterogeneous diseases characterized by the presence of abnormal myeloblasts in the bone marrow and significant chromosomal alterations which often give rise to balanced translocations. Cytogenetics, together with patients' age and prognostic factors affect the choice of treatment. In patients with good-risk cytogenetics such as the translocations t(8;21), intensive chemotherapy including doxorubicin and Ara-C has shown good response rates with overall survival at 5 years of 65%. In APL (Acute Promyelocitic Leukaemia) patients, carrying the translocation t(15;17), the treatment differs from the standard AML recommendation. Indeed ATRA, all-trans retinoic acid, alongside the standard chemotherapy, represents the elective treatment for this specific subgroup with an overall survival at 5 years approaching 80-90% (Sanz and Lo-Coco 2011). In patients with intermediate and poor risk cytogenetics the response decreases to 41% and 14% and the initial chemotherapy regimen (induction) may be followed by a second one (consolidation) or bone marrow transplantation (Grimwade, Walker et al. 1998, Suciu, Mandelli et al. 2003)

Although in the last decades the overall survival of AML patients improved significantly, the majority of them relapse or experience serious treatment-associated complications. Chemotherapy related mortality tend to be even worse in patients older than 60 years. The remission rates for those older than 70 years, which represent the majority of AML patients, are indeed of only 26%. Likewise, a significant proportion of APL patients relapses or shows resistance to the ATRA treatment. Therefore there is an urgent need to design better therapeutic strategies with low or minimal side effects and able to offer efficient alternative to patient resistant to standard therapy.

An ideal cancer therapy would be able to specifically eradicate cancer cells, while sparing normal cells. Synthetic-lethality using poly-(ADP-ribose)-polymerase (PARP) inhibitors have been proven a safe and efficacious treatment for breast and ovarian cancer with mutations of BRCA1/BRCA2, two well-known mediators of Homologous Recombination (HR)-DNA damage repair (DDR) due to the dependence of PARP defective cells on HR (Bryant, Schultz et al. 2005, Farmer, McCabe et al. 2005, Fong, Boss et al. 2009, Tutt, Robson et al. 2010).

Recently, it has been demonstrated that a variety of onco-fusion proteins such as AML1-ETO, PML-RARα, MLL-AF9 and CBFβ-SMMHC produced by the translocations t(8;21), t(15;17), t(9;11) and t(16;16) or inversion inv(16), respectively, may have differential impacts on DDR, genomic instability and induction of senescence. While AML-ETO and PML-RARα repress a variety of genes involved in double strand break (DSB) repair Homologous Recombination pathway (HR) such as BRCA2, RAD50 and RAD51 (Alcalay, Meani et al. 2003, Krejci, Wunderlich et al. 2008) the mechanism behind the genomic instability of MLL-rearranged leukaemia is less well-known and has been related to replication stress (Takacova, Slany et al. 2012).

Accordingly, there is still a need for improved methods for treating AML. In particular, there is a need for alternative treatments for subjects who are resistant to the standard therapies, or who relapse or experience severe side effects. Thus methods which target improved therapeutic agents specifically to sensitive sub-groups of patients are required. The present invention seeks to address these problems by providing methods for identifying AML patients who will respond to treatment with a PARP inhibitor.

### SUMMARY OF THE INVENTION

Accordingly, in one aspect the present invention provides a method for predicting responsiveness of a subject to a poly-(ADP-ribose)-polymerase (PARP) inhibitor for treating acute myeloid leukaemia (AML), the method comprising determining whether a chromosomal abnormality selected from t(8;21), t(15;17), t(16;16) and inv(16) is present in a sample obtained from the subject; wherein the presence of the chromosomal abnormality is indicative of responsiveness of the subject to the PARP inhibitor for treating AML.

In one embodiment, the PARP inhibitor is selected from olaparib (4-[(3-[(4-cyclopropylcarbonyl)piperazin-4-yl]carbonyl)-4-fluorophenyl]methyl(2H)-phthalazin-1-one), veliparib (2-((R)-2-methylpyrrolidin-2-yl)-1H-benzimidazole-4-carboxamide), CEP-8983 (11-methoxy-4,5,6,7-tetrahydro-1H-cyclopenta[a]pyrrolo[3,4-c]carbazole-1,3(2H)-dione) or a prodrug thereof (e.g. CEP-9722), rucaparib (8-Fluoro-2-{4-[(methylamino)methyl]phenyl}-1,3,4,5-tetrahydro-6H-azepino[5,4,3-cd]indol-6-one), E7016 (10-((4-Hydroxypiperidin-1-yl)methyl)chromeno-[4,3,2-de]phthalazin-3(2H)-one), BMN-673 ((8S,9R)-5-fluoro-8-(4-fluorophenyl)-9-(1-methyl-1H-1,2,4-triazol-5-yl)-8,9-dihydro-2H-pyrido[4,3,2-de]phthalazin-3(7H)-one), and INO-1001 (4-phenoxy-3-pyrrolidin-1-yl-5-sulfamoyl-benzoic acid), and analogues and derivatives thereof. Preferably the PARP inhibitor is olaparib or veliparib, more preferably olaparib.

In one embodiment, the chromosomal abnormality is the translocation t(8;21). Typically this chromosomal translocation results in expression of a fusion protein comprising acute myeloid leukemia-1 transcription factor and eight-twenty-one corepressor (AML1-ETO).

In another embodiment, the chromosomal abnormality is the translocation t(15;17). Typically this chromosomal translocation results in expression of a fusion protein comprising promyelocytic leukemia protein and retinoic acid receptor alpha (PML-RARα).

In another embodiment, the chromosomal abnormality is the translocation t(16;16). Typically this chromosomal translocation results in expression of a fusion protein comprising core binding factor β and smooth muscle myosin heavy chain (CBFβ-SMMHC).

In another embodiment, the chromosomal abnormality is the inversion inv(16). Typically this chromosomal abnormality results in expression of a fusion protein comprising core binding factor β and smooth muscle myosin heavy chain (CBFβ-SMMHC).

In one embodiment, the sample is derived from bone marrow or blood.

In a further aspect, the present invention provides a method for treating a subject for acute myeloid leukaemia (AML), the method comprising: (i) predicting responsiveness of the subject to a poly-(ADP-ribose)-polymerase (PARP) inhibitor by a method as defined above; and (ii) treating the subject with a PARP inhibitor if the subject is predicted to be responsive thereto.

In one embodiment, the PARP inhibitor which is administered to the subject is selected from olaparib, veliparib, CEP-8983 or a prodrug thereof (e.g. CEP-9722), rucaparib, E7016, BMN-673, INO-1001, analogues and derivatives thereof. Preferably the PARP inhibitor is olaparib or veliparib, more preferably olaparib.

In one embodiment, the PARP inhibitor is administered to a subject having the chromosomal translocation t(8;21) in combination with a chemotherapeutic agent. Preferably the chemotherapeutic agent is selected from cytarabine (ara-C) and/or an anthracycline.

In another embodiment, the PARP inhibitor is administered to a subject having the chromosomal translocation t(8;21) who previously failed to respond adequately to chemotherapy. Preferably the chemotherapy comprises administration of a chemotherapeutic agent selected from cytarabine (ara-C) and/or an anthracyline.

In another embodiment, the PARP inhibitor is administered to a subject having the chromosomal translocation t(15;17) in combination with a chemotherapeutic agent, preferably all-trans-retinoic acid (ATRA) and/or an anthracycline.

In another embodiment, the PARP inhibitor is administered to a subject having the chromosomal translocation t(15;17) who previously failed to respond to chemotherapy. Preferably the chemotherapy comprises treatment with all-trans-retinoic acid (ATRA) and/or an anthracycline.

In another embodiment, the PARP inhibitor is administered to a subject having the chromosomal translocation t(16;16) in combination with a chemotherapeutic agent. Preferably the chemotherapeutic agent is selected from cytarabine (ara-C) and/or an anthracycline.

In another embodiment, the PARP inhibitor is administered to a subject having the chromosomal translocation t(16;16) who previously failed to respond adequately to chemotherapy. Preferably the chemotherapy comprises administration of a chemotherapeutic agent selected from cytarabine (ara-C) and/or an anthracyline.

In another embodiment, the PARP inhibitor is administered to a subject having the inversion inv(16) in combination with a chemotherapeutic agent. Preferably the chemotherapeutic agent is selected from cytarabine (ara-C) and/or an anthracycline.

In another embodiment, the PARP inhibitor is administered to a subject having the inversion inv(16) who previously failed to respond adequately to chemotherapy. Preferably the chemotherapy comprises administration of a chemotherapeutic agent selected from cytarabine (ara-C) and/or an anthracyline.

In some embodiments, the anthracycline is daunorubicin or doxorubicin.

In one embodiment, the subject is suffering from relapsed AML.

In another embodiment, the subject is unsuitable for a hematopoietic stem cell transplant.

In a further aspect, the present invention provides a poly-(ADP-ribose)-polymerase (PARP) inhibitor for use in treating acute myeloid leukaemia (AML) in a subject, wherein the subject has a chromosomal abnormality selected from t(8;21), t(15;17), t(16;16) and inv(16).

In one embodiment, the PARP inhibitor is selected from olaparib, veliparib, CEP-8983 or a prodrug thereof (e.g. CEP-9722), rucaparib, E7016, BMN-673, INO-1001 and analogues and derivatives thereof. Preferably the PARP inhibitor is olaparib or veliparib, more preferably olaparib.

In one embodiment, the subject has the chromosomal translocation is t(8;21). Preferably the subject is resistant (e.g. non-responsive or shows an inadequate response) to treatment with cytarabine (ara-C) and/or an anthracyline.

In one embodiment, the subject has the chromosomal translocation is t(16;16). Preferably the subject is resistant (e.g. non-responsive or shows an inadequate response) to treatment with cytarabine (ara-C) and/or an anthracyline.

In one embodiment, the subject has the inversion inv(16). Preferably the subject is resistant (e.g. non-responsive or shows an inadequate response) to treatment with cytarabine (ara-C) and/or an anthracyline.

In another embodiment, the subject has the chromosomal translocation t(15;17). Preferably the subject is resistant (e.g. non-responsive or inadequately responsive) to treatment with all-trans-retinoic acid (ATRA) and/or an anthracycline.

In another embodiment, the AML comprises relapsed AML, i.e. the PARP inhibitor is for use in treating relapsed AML in a subject.

In another embodiment, the subject is unsuitable for a hematopoietic stem cell transplant.

In a further aspect, the present invention provides a pharmaceutical combination comprising (i) a poly-(ADP-ribose)-polymerase (PARP) inhibitor and (ii) a chemotherapeutic agent and/or all-trans-retinoic acid (ATRA); for simultaneous, separate or sequential use in treating acute myeloid leukaemia (AML) in a subject.

In one embodiment, the subject has a chromosomal abnormality selected from t(8;21), t(15;17) and inv(16).

In one embodiment, the PARP inhibitor is selected from olaparib, veliparib, CEP-8983 or a prodrug thereof (e.g. CEP-9722), rucaparib, E7016, BMN-673, INO-1001 and analogues and derivatives thereof. Preferably the PARP inhibitor is olaparib or veliparib, more preferably olaparib.

In one embodiment, the chemotherapeutic agent is selected from cytarabine (ara-C) and/or an anthracycline. Preferably the anthracycline is daunorubicin or doxorubicin.

### BRIEF DESCRIPTION OF THE DRAWINGS

FIG. 1 PARPi titration. A) PARPi titration in normal bone marrow c-kit cells grown in methylcellulose for 7 days. Data representative of three independent experiments are shown ±SD. Paired two-tailed t test was performed between untreated and olaparib treated cells for each condition *p<0.05. B) Non linear regression dose response curve, obtained by logarithmic transformation of the data presented in the panel A. C) Colony morphology (contrast microscopy magnification 40×). Representative pictures are shown.

FIG. 2 AML1-ETO and PML-RARα leukemic cells are sensitive to PARPi. A) Representative scheme of experimental procedures. B) Non linear regression dose response curve. Normal bone marrow c-kit cells and pre-leukaemic cells expressing the oncofusion proteins AML1-ETO, E2A-PBX, MLL-AF9 and PML-RARα were grown in methylcellulose for 7 days with escalating doses of PARPi. Data representative of three independent experiments are shown. C) Percentage of survival of preleukemic cells to PARPi. The number of colonies surviving to seven days incubation with PARPi treatment was normalized against the untreated control. Data representative of six independent experiments are shown ±SD. 2-way Anova test was performed between untreated and Olaparib treated cells for each condition *p<0.05. D) Colony morphology (phase contrast microscopy, magnification 40×). Representative pictures are shown. E) Efficiency of PARP1 Knock down (KD) in NIH3T3 cells infected with retroviral vectors expressing shRNA against mouse PARP1. qPCR data showing expression of PARP1 mRNA in NIH3T3 after infection with sh-mPARP1. Data representative of two independent experiments are shown ±SD. Paired two-tailed t test was performed between empty vector and i) scramble, ii) sh-PARP1-A and iii) sh-PARP1-D*p<0.05. F) Efficiency of PARP1 by Western Blot. Western Blot data showing protein level of PARP1 in NIH3T3 after infection with sh-mPARP1. G) Efficiency of PARP1 KD in leukemic cells infected with retroviral vectors expressing shRNA against mouse PARP1. qPCR data showing expression of PARP1 mRNA in leukemic cells after infection with sh-mPARP1. Data representative of three independent experiments are shown ±SD. 2-way Anova test was performed between empty vector and i) scramble, ii) sh-PARP1-A and iii) sh-PARP1-D*p<0.05, **p<0.01, ***p<0.001. H) Relative number of colonies arisen from leukemic cells transformed by indicated oncoproteins after shPARP1-mediated KD. Bone marrow cells were co-transduced with retroviral vectors carrying the oncogene of interest and empty vector or sh-scramble or shPARP1. The number of colonies was normalized against the empty vector control. Data representative of three independent experiments are shown ±SD. 2-way Anova test was performed between empty vector and i) scramble, ii) sh-PARP1-A and iii) sh-PARP1-D*p<0.05, **p<0.01, ***p<0.001. I) Colony morphology from phase contrast microscopy (magnification 40×). Representative pictures are shown. J) Leukaemic cells expressing the oncofusion proteins AML1-ETO, E2A-PBX, MLL-AF9 and PML-RARα were grown in methylcellulose for 7 days with Olaparib and Veliparib (1 μM). The number of colonies after PARPi treatment was normalized against the untreated control. Data representative of three independent experiments are shown ±SD. 2-way Anova test was performed between untreated and Olaparib treated cells for each condition *p<0.05. K) Colony morphology (phase contrast microscopy, magnification 20×). Representative pictures are shown. L) Human cell lines Kasumi, NB4 and THP1 grown in methylcellulose for 7 days with Olaparib 1-5 μM. The number of colonies after PARPi treatment was normalized against the untreated control. Data representative of three independent experiments are shown ±SD. 2-way Anova test was performed between untreated and Olaparib treated cells for each condition *p<0.05. M) Colony morphology (phase contrast microscopy, magnification 20×). Representative pictures are shown. N) Human cell lines NB4, NB4-LR2 and NB4-MR2 were grown in methylcellulose for 7 days with Olaparib 5 μM. The number of colonies after PARPi treatment was normalized against the untreated control. Data representative of three independent experiments are shown ±SD. 2-way Anova test was performed between untreated and Olaparib treated cells for each condition *p<0.05. O) Colony morphology (phase contrast microscopy, magnification 20×). Representative pictures are shown.

FIG. 3 PARPi treatment induces cell cycle arrest in AML-ETO and PML-RARα leukemic cells resulting in differentiation, apoptosis and senescence. A) Flow cytometry analysis confirming no difference in number of progenitor cells (c-kit positive) and differentiated myeloid cells (Mac1, Gr1 double positive cells) in E2A-PBX and MLL-AF9 leukemic cells before and after treatment with PARPi. B) Giemsa-MacGrunwald staining of leukaemic cells showing myeloid differentiation in AML1-ETO and PML-RARα leukemic cells treated with PARPi (Bright field microscopy, magnification 40×). Arrows indicate differentiated cells. C) PARPi significantly affects the survival and proliferation of AML-ETO and PML-RARα leukaemic cells. Cell proliferation was evaluated using MTS assay after 48 hrs of culture with escalating concentration of PARPi. The number of cells after PARPi treatment was normalized against the untreated control. Data representative of ten independent experiments are shown ±SD. D) Immunoblotting of p53, p21 and p27 in leukaemic cells upon PARPi treatment for 6 or 24 hours. E) PARPi induces apoptosis in PML-RARα leukaemic cells. The graph shows the percentage of Annexin V positive cells upon PARPi treatment for 24 or 48 hrs. Data representative of five independent experiments are shown ±SD. 2-way Anova test was performed between untreated and Olaparib treated cells for each condition at each time point, **p<0.01. F) RT-qPCR data of p53 and p53 target genes (p21 and Bax) in leukaemic cells before and after treatment with PARPi. Data representative of four independent experiments are shown ±SD*p<0.05. G) PARPi induces senescence in AML-ETO leukaemic cells. β Galactosidase staining, representative pictures (phase contrast microscopy, magnification 40×). H) The graph shows the percentage of β Galactosidase positive cells. Data representative of three independent experiments are shown ±SD. 2-way Anova test was performed between untreated and Olaparib treated cells for each condition at each time point. I) RT-qPCR data of p16 in leukaemic cells before and after treatment with PARPi. Data representative of three independent experiments are shown ±SD**p<0.01. J) Immunoblotting of p16 in leukaemic cells upon PARPi treatment for 6-24 hours.

FIG. 4 AML-ETO and PML-RARα show multiple defects in DNA repair.

A) Immunofluorescence microscopy of PARPi induced yH2AX foci. Merge are shown with nuclei in blue, yH2AX foci in green and Rad51 foci in red (magnification 100×). B) The percentage of cells with more than 6 yH2AX (black bars) and Rad51 (white bars) foci±SD in untreated condition is shown (n=4*p<0.05). Paired two-tailed t-test was performed between i) AML1-ETO and E2A-PBX, ii) MLL-AF9 and E2A-PBX, and iii) PML-RARα and E2A-PBX. Black asterisks refer to differences in the number of yH2AX foci. Red asterisks refer to differences in the number of Rad51 foci. C) The percentage of cells with more than 6 yH2AX (black bars) and Rad51 (white bars) foci±SD under PARPi treatment for 6 hrs is shown (n=4*p<0.05). Paired two-tailed t-test was performed between i) AML1-ETO and E2A-PBX, ii) MLL-AF9 and E2A-PBX, and iii) PML-RARα and E2A-PBX for both yH2AX and Rad51 sets of data. D) The percentage of cells with yH2AX/Rad51 ratio>2 is shown (n=4*p<0.05). E) RT-qPCR data of Rad51, Xrcc2, Brca1 and Brca2 expression in leukemic cells. Data representative of four independent experiments are shown ±SD*p<0.05, **p<0.005, ***p<0.0005. F) Western blot analysis of Rpa1 and Rad51 in leukaemic cells. G) Box-plot microarray detection of mRNA of BRCA1, BRCA2, RAD51 and XRCC2 expression in patients affected by AML carrying the translocation AML1-ETO (n=22), APL (n=19) or MLL (n=16). Circles represent outliners. Statistical test was performed between expression values in AML1-ETO and MLL or APL and MLL*p<0.050, ***p<0.0011. H) Colony forming efficiency as indicative of DSB repair is shown. Repair efficiency is assessed as the total number of bacterial colonies obtained per transformation and expressed as mean±standard deviation. Paired two-tailed t-test was performed between i) AML1-ETO and E2A-PBX, ii) MLL-AF9 and E2A-PBX and iii) PML-RARα and E2A-PBX. I) Percentage of misrepair in leukaemic cells. Misrepair is calculated as the fraction of white colonies in total (blue and white) colonies, expressed as mean±standard deviation. Paired two-tailed t-test was performed between i) AML1-ETO and E2A-PBX, ii) MLL-AF9 and E2A-PBX and iii) PML-RARα and E2A-PBX.

FIG. 5 PARPi treatment prolongs the survival in mice models of human leukaemia driven by AML1-ETO and PML-RARα oncogenes.

A) Kaplan-Meier survival curve relative to NSG mice injected with NB4 human cells. NB4 human cells expressing the oncogene PML-RARα were injected intravenously in NSG mice and distributed in two groups. The control group received daily injection of DMSO in 10% (2-Hydroxypropyl)-β-cyclodextrin (HBC), whereas the treated group received daily injection of Olaparib 25 mg/kg in 10% HBC. Treatment was performed for two weeks. Mice were monitored daily until the development of symptoms of leukaemia, when bone marrow (B), spleen (C) and liver (D) were harvested, processed, stained with PE-conjugated antibody against human CD33 and analysed by FACS, to quantify the human cell engraftment in these organs. E) Kaplan-Meier survival curve relative to NSG mice injected with Kasumi human cells. Kasumi human cells expressing the oncogene AML1-ETO were injected intra-femur in NSG mice and distributed in two groups. The control group received daily injection of DMSO in 10% HBC, whereas the treated group received daily injection of Olaparib 25 mg/kg in 10% HBC. Treatment was performed for two weeks. Mice were monitored daily until the development of symptoms of leukaemia, when bone marrow (F), spleen (G) and liver (H) were harvested, processed, stained with PE-conjugated antibody against human CD33 and analysed by FACS, to quantify the human cell engraftment in these organs. I) and J Kaplan-Meier survival curves of FVB and C57Bl/6 mice injected with M4-PML-RARα and MLL-AF9 cells, respectively. ATRA resistant M4-PML-RARα cells were injected intravenously in FVB mice and distributed in three groups. The control group received daily injection of DMSO in 10% HBC, whereas the treated groups received daily injection of Olaparib 50 mg/kg or ATRA 1 mg/Kg in the same solvent Treatment was performed for two weeks. Mice were monitored daily until the development of symptoms of leukaemia, when bone marrow (K), spleen (L) and liver (M) were harvested, processed, stained with PE-conjugated antibody against mouse Gr1 and analysed by FACS, to quantify the human cell engraftment in these organs. K), L) and M) represent the relative engraftment of M4-PML-RARα in bone marrow, spleen and liver, respectively. Data representative of the average of three independent experiments are shown ±SD. Unpaired one-tailed t test was performed between vehicle and olaparib or vehicle and ATRA*p<0.05, **p<0.005, ***p<0.0005

### DETAILED DESCRIPTION OF THE INVENTION

The present disclosure demonstrates that DNA repair inhibitors, such as Poly ADP-ribose Polymerase inhibitors (PARPi), are effective agents to specifically eradicate leukaemia cells sustained by certain onco-fusion proteins such as AML1-ETO and PML-RARα. Interestingly MLL-AF9 cells were found to be resistant to PARPi suggesting potential back-up pathways, which may offset the effects associated with DDR and allow MLL-rearranged leukaemia to cope with genomic instability. These results show that although genomic instability is a common feature of leukaemia, not all AML patients are suitable for PARPi treatment. Thus in aspects of the present invention, cytogenetics results may be used to identify sub-groups of AML patients who will be sensitive to PARPi.

### Predicting Responsiveness

The present method relates to predicting responsiveness of a subject to a PARP inhibitor. By this it is meant that the method may be used to determine whether or not administration of a PARP inhibitor to the subject is likely to provide a clinical benefit. The benefit may be, for example, in terms of a remission of the disease, an alleviation of one or more signs or symptoms of the disease, a delay in progression of disease or an increase in survival time.

In particular, in preferred embodiments responsiveness of the subject and/or clinical benefit may be measured in terms of disease remission or an increased survival time following treatment with the PARP inhibitor (e.g. olaparib). For instance, in one embodiment a clinical benefit may be indicated by a decreased incidence of one or more clinical signs of AML such as abnormal white blood cell counts (e.g. leukocytosis or leukopenia), decreased numbers of normal leukocytes, increased numbers of leukemic myeloblasts, neutropenia, anemia and/or thrombocytopenia. In other embodiments, a benefit may be indicated by a decrease in one or more symptoms of AML selected from fatigue, shortness of breath, bruising or bleeding, and increased risk of infection.

### Subjects

In one embodiment, the subject is a human. In a preferred embodiment the subject is an adult human, although in some embodiments the method may be performed on a child or infant. In other embodiments, the subject may be aged 50 years or over, 60 years or over, or 70 years or over.

The subject is typically suffering from AML, or suspected to be suffering from AML. The method may be used, for instance, to select a personalized treatment protocol for the subject. The method may be performed before the subject has received (e.g. a PARP inhibitor) treatment for AML, or after treatment has already commenced, for instance in order to decide whether to continue treating the subject with a PARP inhibitor-based therapy.

### Poly ADP-Ribose Polymerase Inhibitors (PARPi)

PARP1 is a nuclear protein that senses single and double strand DNA breaks (SSB and DSB) catalyzing the addition of poly ADP ribose to itself, histones, topoisomerase I, DNA protein kinase (DNA-PK), XRCC1 and other proteins involved in DNA repair (Brightwell and Shall 1971, Krishnakumar and Kraus 2010). When PARP-1 is inhibited, it can be trapped on the SSB intermediate, preventing the ligation step and inducing accumulation of SSB that then collapse replication forks in DSB (Bryant, Schultz et al. 2005, Farmer, McCabe et al. 2005, Helleday, Petermann et al. 2008). Seventeen PARP proteins have been identified so far, but not all of them are enzymatically active (Rouleau, Patel et al. 2010).

In general, the PARP inhibitor may be any agent that can inhibit the activity of PARP, for example, any one or more of PARP 1-17. Preferably the agent is a small molecule inhibitor. In one embodiment, the PARP inhibitor inhibits the activity of PARP1 and/or PARP2. PARP inhibitors may be selected from compounds having PARP inhibitory activity and one of the following general structures: nicotinamides, such as 5-methyl nicotinamide and O-(2-hydroxy-3-piperidino-propyl)-3-carboxylic acid amidoxime, and analogues and derivatives thereof; benzamides, including 3-substituted benzamides such as 3-aminobenzannide, 3-hydroxybenzamide 3-nitrosobenzamide, 3-methoxybenzamide and 3-chloroprocainamide, and 4-aminobenzamide, 1,5-di[(3-carbamoylphenyl)aminocarbonyloxy]pentane, and analogues and derivatives thereof; isoquinolinones and dihydroisoquinolinones, including 2H-isoquinolin-1-ones, 3H-quinazolin-4-ones, 5-substituted dihydroisoquinolinones such as 5-hydroxy dihydroisoquinolinone, 5-methyl dihydroisoquinolinone, and 5-hydroxy isoquinolinone, 5-amino isoquinolin-1-one, 5-dihydroxyisoquinolinone, 3,4 dihydroisoquinolin-1(2H)-ones such as 3,4 dihydro-5-methoxy-isoquinolin-1(2H)-one and 3, 4 dihydro-5-methyl-1(2H)isoquinolinone, isoquinolin-1(2H)-ones, 4,5-dihydro-imidazo[4,5,1-ij]quinolin-6-ones, 1,6,-naphthyridine-5(6H)-ones, 1,8-naphthalimides such as 4-amino-1,8-naphthalimide, isoquinolinone, 3,4-dihydro-5-[4-1(1-piperidinyl)butoxy]-1(2H)-isoquinolinone, 2,3-dihydrobenzo[de]isoquinolin-1-one, 1-11b-dihydro-[2H]benzopyrano[4,3,2-de]isoquinolin-3-one, and tetracyclic lactams, including benzpyranoisoquinolinones such as benzopyrano[4,3,2-de]isoquinolinone, and analogues and derivatives thereof; benzimidazoles and indoles, including benzoxazole-4-carboxamides, benzimidazole-4-carboxamides, such as 2-substituted benzoxazole 4-carboxamides and 2-substituted benzimidazole 4-carboxamides such as 2-aryl benzimidazole 4-carboxamides and 2-cycloalkylbenzimidazole-4-carboxamides including 2-(4-hydroxphenyl)benzimidazole 4-carboxamide, quinoxalinecarboxamides, imidazopyridinecarboxamides, 2-phenylindoles, 2-substituted benzoxazoles, such as 2-phenyl benzoxazole and 2-(3-methoxyphenyl)benzoxazole, 2-substituted benzimidazoles, such as 2-phenyl benzimidazole and 2-(3-methoxyphenyl)benzimidazole, 1,3,4,5 tetrahydro-azepino[5,4,3-cd]indol-6-one, azepinoindoles and azepinoindolones such as 1,5 dihydro-azepino[4,5,6-cd]indolin-6-one and dihydrodiazapinoindolinone, 3-substituted dihydrodiazapinoindolinones such as 3-(4-trifluoromethylphenyl)-dihydrodiazapinoindolinone, tetrahydrodiazapinoindolinone and 5,6,-dihydroimidazo[4,5,1-j,k][1,4]benzodiazopin-7(4H)-one, 2-phenyl-5,6-dihydro-imidazo[4,5,1-jk][1,4]benzodiazepin-7(4H)-one and 2,3, dihydro-isoindol-1-one, and analogues and derivatives thereof; phthalazin-1(2H)-ones and quinazolinones, such as 4-hydroxyquinazoline, phthalazinone, 5-methoxy-4-methyl-1(2) phthalazinones, 4-substituted phthalazinones, 4-(1-piperazinyl)-1(2H)-phthalazinone, tetracyclic benzopyrano[4,3,2-de]phthalazinones and tetracyclic indeno[1,2,3-de]phthalazinones and 2-substituted quinazolines, such as 8-hydroxy-2-methylquinazolin-4-(3H) one, tricyclic phthalazinones and 2-aminophthalhydrazide, and analogues and derivatives thereof; isoindolinones and analogues and derivatives thereof; phenanthridines and phenanthridinones, such as 5[H]phenanthridin-6-one, substituted 5[H]phenanthridin-6-ones, especially 2-, 3-substituted 5[H]phenanthridin-6-ones and sulfonamide/carbamide derivatives of 6(5H)phenanthridinones, thieno[2,3-c]isoquinolones such as 9-amino thieno[2,3-c]isoquinolone and 9-hydroxythieno[2,3-c]isoquinolone, 9-methoxythieno[2,3-c]isoquinolone, and N-(6-oxo-5,6-dihydrophenanthridin-2-yl]-2-(N,N-dimethylamino}acetamide, substituted 4,9-dihydrocyclopenta[lmn]phenanthridine-5-ones, and analogues and derivatives thereof; benzopyrones such as 1,2-benzopyrone 6-nitrosobenzopyrone, 6-nitroso 1,2-benzopyrone, and 5-iodo-6-aminobenzopyrone, and analogues and derivatives thereof; unsaturated hydroximic acid derivatives such as O-(3-piperidino-2-hydroxy-1-propyl)nicotinic amidoxime, and analogues and derivatives thereof; and pyridazines, including fused pyridazines and analogues and derivatives thereof.

Early generation PARPi had little specificity for individual PARPs and very high IC50 (half-maximal inhibitory concentration), driving significant off target effects. In contrast, olaparib is an oral competitive PARPi targeting the nicotinamide binding pocket of PARP1 and PARP2 (Wahlberg, Karlberg et al. 2012). Developed by Kudos Pharmaceuticals and later by Astra Zeneca, its improved chemical structure increases its specificity and affinity to PARP1 and PARP2.

Olaparib is one of the first PARPi to enter clinical trials and it has already been tested in phase I and phase II trials including breast, prostate and ovarian cancer patients carrying mutations in BRCA1 or BRCA2 genes, showing anti-tumour effect and side effects of grade 1 or 2 (Fong, Boss et al. 2009, Tutt, Robson et al. 2010). Tested in a phase II clinical trial in an untargeted population of ovarian cancer, Olaparib failed to show overall survival benefit compared to chemotherapy. Based on these results, in 2011 AstraZeneca decided not to, as previously planned, pursue a phase III clinical trial in hereditary BRCA1 and BRCA2 associated breast cancer, a controversial decision considering that these patients are the strongest candidates for PARPi. The results of the clinical trial in untargeted populations of ovarian cancer highlighted the importance of identifying patients carrying specific mutations who may benefit from the treatment. More recently, on April 2013 Astrazeneca announced a new Phase III trial of Olaparib for patients with BRCA mutated ovarian cancer, which is due in the end of the year. Olaparib and other PARPi are continuing to advance through clinical trials (see a complete list below), showing promising results in targeted populations of patients with “BRCAness phenotype” (Turner, Tutt et al. 2004).
Astra Zeneca: Olaparib (AZD2281) currently in phase I/II as single agent or combination with chemotherapy in various cancer types including breast, ovarian and colorectal cancers. Currently recruiting ovarian cancer patients carrying BRCA mutation for a phase III.
Abbott: Veliparib (ABT-888) currently in phase I/II in combination with chemotherapy or radiation in various metastatic or unresectable solid tumours including breast, ovarian and colorectal cancer, glioblastoma and melanoma or non-Hodgkin lymphoma; phase II for metastatic melanoma and breast cancer; currently recruiting prostate cancer patients carrying BRCA mutation for a phase III.
Cephalon: CEP-9722 currently in phase I/II in combination with temozolomide in advanced solid tumors and lymphoma. In phase III for non-small cell lung cancer.
Clovis Oncology: Rucaparib (AG014699, PF-01367338) currently in phase I/II in BRCA1/2 mutant cancers and solid tumors; phase III for metastatic ovarian and breast cancer.
Eisai: E7016 currently in phase I in combination with temozolomide in advanced solid tumours.
BioMarin Pharmaceutical: BMN-673 currently in phase I in solid tumours and advanced haematological cancer.
Inotek: INO-1001 currently in phase I in combination with temozolomide in melanoma.
Sanofi-Aventis: Iniparib (BSI-201) currently in phase III in combination with gemcitabine and carbpoplatin in breast and lung cancers; phase I/II: single agent or in combination with chemotherapy in various cancer types including glioma and ovarian cancers; Failed phase III clinical trial for triple negative breast cancer.
Tesaro Inc.: MK4827 currently in phase I in advanced solid tumours or haematological disorders.

Accordingly, in embodiments of the present invention a PARP inhibitor such as one of those described above may be used. Preferably the PARP inhibitor shows inhibition of at least PARP1, e.g. with an IC50 of 100 μM or below, 10 μM or below, or 1 μM or below. Typically PARP inhibitors induce double strand breaks in DNA and cell death in cells in which homologous recombination is inactive (for instance due to mutations in DNA repair genes). Thus PARP inhibitors can be identified using corresponding cellular assays, including as described in the examples below.

In embodiments of the present invention, the PARP inhibitor is preferably selected from olaparib (AZD2281, 4-[(3-[(4-cyclopropylcarbonyl)piperazin-4-yl]carbonyl)-4-fluorophenyl]methyl(2H)-phthalazin-1-one), veliparib (ABT-888, 2-((R)-2-methyl pyrrolidin-2-yl)-1H-benzimidazole-4-carboxamide), CEP-9722 (a prodrug which is converted to 11-methoxy-4,5,6,7-tetrahydro-1H-cyclopenta[a]pyrrolo[3,4-c]carbazole-1,3(2H)-dione (CEP-8983)), rucaparib (AG014699, 8-Fluoro-2-{4-[(methylamino)methyl]phenyl}-1,3,4,5-tetrahydro-6H-azepino[5,4,3-cd]indol-6-one), E7016 (10-((4-Hydroxypiperidin-1-yl)methyl)chromeno[4,3,2-de]phthalazin-3(2H)-one), BMN-673 ((8S,9R)-5-fluoro-8-(4-fluorophenyl)-9-(1-methyl-1H-1,2,4-triazol-5-yl)-8,9-dihydro-2H-pyrido[4,3,2-de]phthalazin-3(7H)-one), and INO-1001 (4-phenoxy-3-pyrrolidin-1-yl-5-sulfamoyl-benzoic acid), and analogues and derivatives thereof. Preferably the PARP inhibitor is olaparib or veliparib, more preferably olaparib.

Further suitable PARP inhibitors and assays for determining PARP inhibition are disclosed in, for example, U.S. Pat. No. 7,041,675, WO07/041357, WO2003/057699 U.S. patent Ser. No. 06/444,676; US 2006/0229289; US2006/0063926; WO2006/033006; WO2006/033007; WO03/051879; WO2004/108723; WO2006/066172; WO2006/078503; US2007/0032489; WO2005/023246; WO2005/097750; WO2005/123687; WO2005/097750; U.S. Pat. No. 7,087,637; U.S. Pat. No. 6,903,101; WO2007/0011962; US 2007/0015814; WO2006/135873; US2007/0072912; WO2006/065392; WO2005/012305; WO2005/012305; EP412848; EP453210; EP454831; EP879820; EP879820; WO030805; WO03/007959; U.S. Pat. No. 6,989,388; US2006/0094746; EP1212328; WO2006078711; U.S. Pat. No. 0,6426,415; U.S. patent Ser. No. 06/514,983; EP1212328; US2004/0254372; US2005/0148575; US2006/0003987; U.S. patent Ser. No. 06/635,642; WO2001/16137; WO2004/105700; WO03/057145; WO2006/078711; WO2002/044157; US2005/6924284; WO2005/112935; US2004/6828319; WO2005/054201; WO2005/054209; WO2005/054210; WO2005/058843; WO2006/003146; WO2006/003147; WO2006/003148; WO2006/003150; WO2006/003146; WO2006/003147; US2007/0072842; U.S. patent Ser. No. 05/587,384; US2006/0094743; WO2002/094790; WO2004/048339; EP1582520; US2006/0004028; WO2005/108400; U.S. Pat. No. 6,964,960; WO2005/0080096; WO2006/137510; US2007/0072841; WO2004/087713; WO2006/046035; WO2006/008119; WO2006/008118; WO2006/042638; US2006/0229289; US2006/0229351; WO2005/023800; WO1991/007404; WO2000/042025; WO2004/096779; U.S. patent Ser. No. 06/426,415; WO02/068407; U.S. patent Ser. No. 06/476,048; WO2001/090077; WO2001/085687; WO2001/085686; WO2001/079184; WO2001/057038; WO2001/023390; WO01/021615; WO2001/016136; WO2001/012199; WO95/024379; WO2002/36576; WO2004/080976; Banasik et al. J. Biol. Chem., 267:3, 1569-75 (1992); Banasik et al. Molec. Cell. Biochem. 138:185-97 (1994); Cosi (2002) Expert Opin. Ther. Patents 12 (7); Southan & Szabo (2003) Curr Med Chem 10 321-340; Annals of Oncology 22 (Supplement 1): i53-i59, 2011; Curr Opin Drug Discov Devel. 2010 September; 13(5):577-86; Mol Cancer Ther (August 2007) 6:2290; and references therein.

### Acute Myeloid Leukaemia

In embodiments of the present invention, the subject is suffering from, or is suspected to be suffering from acute myeloid leukaemia (AML). Acute myeloid leukemia may also be referred to as acute myelogenous leukemia or acute nonlymphocytic leukemia (ANLL). AML is a cancer of the myeloid line of leukocytes, and is characterized by the rapid growth and accumulation of abnormal leukocytes in the bone marrow. AML is the most common acute leukemia affecting adults, and its incidence increases with age.

About half of the subjects suffering from AML have at least one chromosomal abnormality. In particular, the chromosomal translocations t(8;21) and t(15;17) and abnormal 11q23 are associated with production of the oncogenic fusion proteins AML1-ETO, PML-RARα and MLL-AF9 respectively, and each account for around 5 to 10% of adult and childhood cases of AML. In addition, the chromosomal inversion inv16 and the translocation t(16;16), both resulting in the expression of the oncofusion protein CBFβ-SMMHC, are associated with around 5% of AML cases in both adults and children. The remaining AML patients typically show a normal karyotype in cytogenetic studies, or a complex cytogenetic profile, which usually associates with a poor prognosis. Thus in embodiments of the present invention, responsiveness to a PARP inhibitor is typically indicated in around 20 to 25% of all AML patients, i.e. those having a chromosomal abnormality selected from t(8;21), t(15;17), t(16;16) and inv(16).

As used herein, the term acute myeloid leukaemia (AML) includes acute promyelocytic leukemia (APL), which is a sub-type of AML characterized by an abnormal accumulation of immature granulocytes (promyelocytes). APL is also known as acute progranulocytic leukemia, and is typically associated with a chromosomal translocation involving the retinoic acid receptor-alpha gene on chromosome 17 (RARα). In a large proportion of cases of APL, the RARα gene on chromosome 17 is involved in a reciprocal translocation with the promyelocytic leukemia gene (PML) on chromosome 15, a translocation denoted as t(15;17) which results in production of the oncogenic fusion protein PML-RARα.

In further embodiments, the subject may be suffering from a preleukemic blood disorder, e.g. a condition which may develop into AML. Examples of such conditions include myelodysplastic syndrome and myeloproliferative disease.

In some embodiments, the subject is suffering from relapsed AML. A large proportion of AML patients relapse, i.e. one or more clinical signs or symptoms of AML re-appear in the subject after a remission. A remission may be indicated by an alleviation of one or more signs or symptoms of the disease, e.g. a decrease in the number of leukemic myeloblasts or a normalization of other parameters of leukocyte function. Typically a period of remission is induced by an initial phase of therapy, e.g. using one or more standard therapies for AML as described below.

### Sample

The method of the present invention is performed using a sample obtained from the subject. Typically the sample comprises leukocytes, especially those of the myeloid lineage. In preferred embodiments, the sample is derived from peripheral blood or bone marrow. For instance, the sample may comprise blood cells or bone marrow cells obtained from blood or bone marrow smears. In particular, bone marrow cells may be obtained by bone marrow aspiration or biopsy using known techniques.

### Detecting Chromosomal Abnormalities

Embodiments of the present invention may comprise detecting a chromosomal abnormality selected from t(8;21), t(15;17), t(16;16) and inv(16) in the sample from the subject. These abnormalities may be detected using, for example, cytogenetic methods such as karyotyping or fluorescent in situ hybridisation or by Reverse Transcription-Polymerase Chain Reaction (RT-PCR).

Karyotyping refers to chromosome analysis, e.g. of metaphase chromosomes which have been banded using trypsin and histological stains, resulting in unique banding patterns on the chromosomes. Various chromosome-banding techniques may be used, including quinacrine banding (Q-banding), Giemsa banding (G-banding), reverse banding (R-banding), constitutive or centromere banding (C-banding) and nucleolar organizing region stains (NOR stains). High-resolution banding may also be used, e.g. to increase the number of observable bands.

A sample comprising cells from e.g. bone marrow or blood may be cultured using standard cell culture techniques in order to increase the number of observable leukemic myeloblasts. A mitotic inhibitor such as colchicine may then be added to the culture to halt cell division at mitosis. A hypotonic solution may be used to swell the cells and induce spreading of the chromosomes, as well as lysing red blood cells. The cells are then fixed and the banding pattern analysed under a microscope. Typically the presence of particular chromosomal abnormalities can be determined by the presence of corresponding banding patterns in the nuclei of leukemic myeloblasts from the subject.

Alternatively, the chromosomal abnormalities may be detected using fluorescent in situ hybridization (FISH). Fluorescent in situ hybridization utilises a fluorescently labelled polynucleotide probe to hybridize to specific DNA sequences in the chromosomes. FISH may be performed, for example, on bone marrow smears or blood smears as well as uncultured bone marrow aspirates or biopsy samples.

Typically FISH is performed on a microscope slide comprising the blood or bone marrow sample. The DNA probe is allowed to hybridise to complementary sequences present in the sample, after which bound probe is visualised using fluorescence microscopy. In some embodiments a number of leukemic myeloblasts may be analysed to look for the presence of a particular mutation which is specifically bound by the probe. Accordingly, specific chromosomal abnormalities can be detected in the sample from the subject.

More recently a molecular test, the Reverse Transcription-Polymerase Chain Reaction test (RT-PCR) has entered the routine diagnostics of AML and in particular of APL. This test allows to specifically detect the fusion genes associated with leukaemia, e.g. AML1-ETO, PML-RARα, CBF-MYH11. After extracting RNA from the blood or bone marrow of the patients, the RNA is retro-transcribed in cDNA which will be then used as template for the PCR reaction. By using oligonucleotide primers specific for certain sequences, typically spanning the breakpoints, regions of interest can be amplified and further analyzed.

In one embodiment, the chromosomal abnormality which is detected is a translocation, e.g. t(8;21), t(16;16) or t(15;17). The translocation t(8;21) typically results in expression of a fusion protein comprising acute myeloid leukemia-1 transcription factor and eight-twenty-one corepressor (AML1-ETO). The translocation t(15;17) typically results in expression of a fusion protein comprising promyelocytic leukemia protein and retinoic acid receptor alpha (PML-RARα). The translocation t(16;16) results in expression of a fusion protein comprising core binding factor (3 and smooth muscle myosin heavy chain (CBFβ-SMMHC), which is encoded by MYH11.

In another embodiment, the chromosomal abnormality is an inversion, e.g. an inversion on chromosome 16 designated as inv(16) which results in expression of a fusion protein comprising core binding factor β and smooth muscle myosin heavy chain (CBFβ-SMMHC), which is encoded by MYH11. These chromosomal abnormalities may be detected using cytogenetics (e.g. karyotyping or FISH) or RT-PCR as described above.

In the last few years many studies have converged in the identification of biomarkers of HR deficiency, also named “BRCAness”, which could predict PARPi sensitivity (Turner, Tutt et al. 2004). To date, in addition to deficiency of BRCA 1 and BRCA2 (Bryant, Schultz et al. 2005, Farmer, McCabe et al. 2005) new biomarkers of PARPi sensitivity have been identified, including deficiency of PTEN (Mendes-Pereira, Martin et al. 2009, Dedes, Wetterskog et al. 2010) ATM mutation (Schaffner, Stilgenbauer et al. 1999), MRE11 mutation (Wen, Scorah et al. 2008) and FANCF promoter methylation (Olopade and Wei 2003). However the identification of these mutations in solid tumours is not yet part of the routine diagnostics offered to the patients and therefore matching the PARPi treatment to the most appropriate cohort of patients is still a challenge. Instead the methods described above, and in particular karyotyping and FISH, represent routine diagnostics tools used to stratify the AML patients. Therefore the methods of the present invention can be translated very quickly into clinical practice.

The presence of at least one of the above chromosomal abnormalities in the sample is typically indicative of responsiveness of the subject to a PARP inhibitor. In other words, if t(8;21), t(15;17), t(16:16) or inv(16) is detected in the sample, it is likely that administration of a PARP inhibitor to the subject may be of clinical benefit.

### Treating AML

In one embodiment, the method described above may be used in order to select a treatment protocol for an individual subject. For instance, based on whether the subject is predicted to be responsive thereto, a PARP inhibitor may be administered to the subject or an alternative treatment strategy employed.

If the method indicates that the subject is likely to be responsive, in particular embodiments the PARP inhibitor may be provided as a first or second line therapy, either alone or in combination with other agents. In one embodiment, the PARP inhibitor may be used as a first line therapy, i.e. in the treatment of subjects who have not previously been treated for AML. In an alternative embodiment, the PARP inhibitor may be used as a second line therapy, e.g. in the treatment of subjects who fail to respond to a first line therapy, who show severe side-effects to the first line therapy or who relapse following a remission subsequent to the first line therapy.

A typical first line therapy for AML may comprise chemotherapy. Chemotherapy typically refers to treatment with drugs or chemical compounds that target cancer cells. Chemotherapy may involve administration of a chemotherapeutic compound, which may have a cytotoxic or cytostatic effect, or which may induce a cyto-protective autophagy response in the cell. The chemotherapeutic agent may be an agent that induces apoptosis, such as p53-dependent apoptosis, or that induces cell cycle arrest, including p53-dependent cell cycle arrest, in a cell that is abnormally proliferating or cancerous. Commonly used chemotherapeutic agents include DNA damaging agents and genotoxic agents that can activate p53-dependent apoptosis or p53-dependent cell cycle arrest in a proliferating cell. In particular embodiments, the PARP inhibitor may be administered to the subject as a first line therapy in combination with one or more such chemotherapeutic agents, or as a second line therapy following chemotherapy as a first line treatment.

The nature of the treatment protocol may vary depending on the type of chromosomal abnormality detected in the subject. For instance, for subjects having the chromosomal translocation t(8;21), t(16;16) or the inversion inv(16), the standard therapy is typically administration of a chemotherapeutic agent such as cytarabine (ara-C), sometimes in combination with an anthracycline such as daunorubicin or doxorubicin. Thus in one embodiment, the PARP inhibitor may be administered to the subject as a first line therapy either alone or in combination with a chemotherapeutic agent selected from cytarabine (ara-C) and/or an anthracycline. Typically the combination of the PARP inhibitor and the chemotherapeutic agent may be more effective than chemotherapy alone, e.g. the combination may improve remission rates and/or an increase the average time before relapse in treated subjects compared to chemotherapy alone.

In an alternative embodiment, the PARP inhibitor may be administered (e.g. as a second line therapy) to a subject having the chromosomal translocation t(8;21), t(16;16) or the inversion inv(16) who previously failed to respond adequately to the standard therapy, e.g. a chemotherapeutic agent selected from cytarabine (ara-C) and/or an anthracyline. An inadequate response may be indicated by, for example, one or more clinical signs or symptoms of the disease remaining following treatment with the chemotherapeutic agent. For instance, the subject may be non-responsive, inadequately responsive or resistant to treatment with cytarabine (ara-C) and/or an anthracyline. Alternatively, the subject may have relapsed or suffered serious side effects following treatment with the chemotherapeutic agent.

In contrast, for subjects having the chromosomal translocation t(15;17), e.g. a subject suffering from PML, the standard therapy is typically based on all-trans-retinoic acid (ATRA or tretinoin), which may be administered in combination with chemotherapy (e.g. an anthracycline such as daunorubicin or doxorubicin). Thus in one embodiment, the PARP inhibitor may be administered to the subject as a first line therapy, either alone or in combination with ATRA and/or an anthracycline. Typically the combination of the PARP inhibitor with ATRA and/or an anthracycline may be more effective than ATRA and/or an anthracyline alone, e.g. the combination may improve remission rates and/or an increase the average time before relapse in treated subjects compared to the standard therapy alone.

In an alternative embodiment, the PARP inhibitor may be administered (e.g. as a second line therapy) to a subject having the chromosomal translocation t(15;17) who previously failed to respond adequately to the standard therapy, e.g. ATRA and/or an anthracyline (preferably ATRA). An inadequate response may be indicated by, for example, one or more clinical signs or symptoms of the disease remaining following treatment with the chemotherapeutic agent. For instance, the subject may be non-responsive, inadequately responsive or resistant to treatment with ATRA and/or an anthracyline (preferably ATRA). Alternatively, the subject may have relapsed or suffered serious side effects following treatment with the standard therapy.

In further embodiments, the PARP inhibitor may be used to treat subjects who are unsuitable for a hematopoietic stem cell transplant (bone marrow transplant). In some cases, the PARP inhibitor may be used as an alternative therapy to a bone marrow transplant. Hematopoietic stem cell transplants are typically indicated as a second line therapy for AML patients at high risk of relapse, or patients who have relapsed following a remission subsequent to the first line treatment. However, since bone marrow transplantation is not necessarily available for all patients, treatment with a PARP inhibitor provides an alternative therapeutic strategy in such cases.

### Pharmaceutical Formulations and Combinations

The PARP inhibitor may be formulated and administered to a subject in any suitable composition, optionally in combination with a chemotherapeutic agent or ATRA for the treatment of AML.

Administration of the PARP inhibitor in combination with a chemotherapeutic agent (e.g. ara-C or an anthracycline) or ATRA typically means that the administration of the PARP inhibitor occurs in a time period during which the subject is undergoing chemotherapy, for example simultaneously with, overlapping with, or sequentially prior to or following the administration of the chemotherapy. The administration of the PARP inhibitor and the chemotherapy (or ATRA) may each be achieved in one or more discrete treatments or may be performed continuously for a given time period required in order to achieve the desired result.

In particular embodiments, an effective amount of the PARP inhibitor, optionally in combination with a chemotherapeutic agent or ATRA, is administered to the subject. In this context, the term “effective amount” means an amount effective, at dosages and for periods of time necessary to achieve the desired result. For example, the PARP inhibitor may be administered in an amount effective to treat AML by reducing one or more clinical signs or symptoms of the disease, e.g. by inducing a remission.

The PARP inhibitor (or chemotherapeutic agent or ATRA) may be administered to a subject using a variety of techniques. For example, the agent may be administered systemically, which includes by injection including intramuscularly or intravenously, orally, sublingually, transdermally, subcutaneously, internasally. The concentration and amount of the PARP inhibitor to be administered will typically vary, e.g. depending on the particular sub-type of AML, the specific PARP inhibitor that is administered, the mode of administration, and the age and health of the subject.

The PARP inhibitor (or chemotherapeutic agent or ATRA) may be formulated in a pharmaceutical composition together with a pharmaceutically acceptable carrier, excipient or diluent. The compositions may routinely contain pharmaceutically acceptable concentrations of salt, buffering agents, preservatives and various compatible carriers.

The proportion and identity of the pharmaceutically acceptable carrier, excipient or diluent may be determined by the chosen route of administration, compatibility with live cells, and standard pharmaceutical practice. Generally, the pharmaceutical composition will be formulated with components that will not significantly impair the biological properties of the agent. Suitable carriers, excipient and diluents are described, for example, in Remington's Pharmaceutical Sciences (Remington's Pharmaceutical Sciences, Mack Publishing Company, Easton, Pa., USA 1985).

Preferably the PARP inhibitor is administered orally. The PARP inhibitor will normally be administered at a unit dose, for example, from about 20 mg to 1 g of active ingredient. The PARP can be formulated in a conventional tablet for oral administration containing 50 mg, 100 mg, 250 mg or 500 mg of active ingredient. Conveniently the daily oral dose is above 150 mg, for example, in the range 150 to 750 mg, preferably in the range 200 to 500 mg. For a single dosage form, the active ingredients may be compounded with an appropriate and convenient amount of excipients which may vary from about 5 to about 98 percent by weight of the total composition. Dosage unit forms will generally contain about 20 mg to about 500 mg of each active ingredient. However the daily dose will necessarily be varied depending upon the patient treated, the particular route of administration, and the severity of the illness being treated. Accordingly the optimum dosage may be determined by the practitioner who is treating any particular patient.

The invention will now be described by way of example only with reference to the following non-limiting embodiments.

### Examples

### PARPi Titration in Bone Marrow Cells

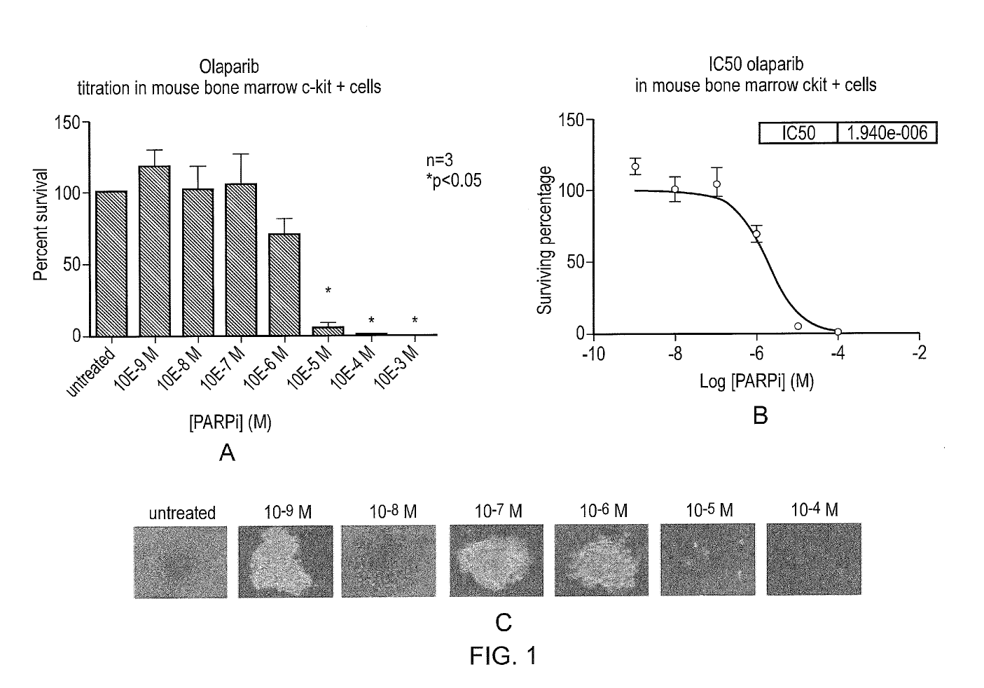

A dose-response titration assay in bone marrow cells in both liquid culture and semisolid medium allowed us to identify the IC50 and the highest Olaparib dose with undetectable or minimal effects on normal bone marrow progenitor cells. Bone marrow cells were treated with escalating doses of Olaparib in methylcellulose. After seven days colonies were counted. The number of colonies obtained in the untreated condition was set as the top value (100%). The bottom value (0) was calculated as the lowest concentration resulting in no colonies. The data where then analyzed in a nonlinear regression dose-response curve (FIG. 1). IC50 for Olaparib in bone marrow c-kit+ cells was 1.9 uM, indicating that Olaparib 500 nM-1 uM could be safely used without harming bone marrow cells.

### AML1-ETO and PML-RARα Leukaemic Cells are Sensitive to PARPi.

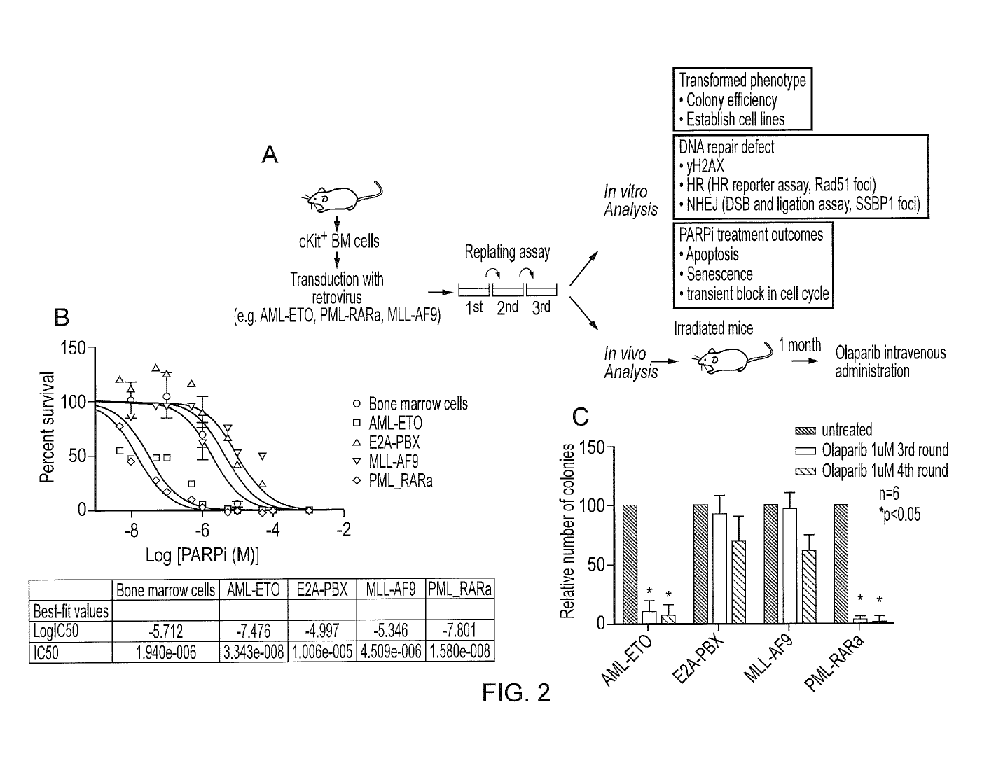

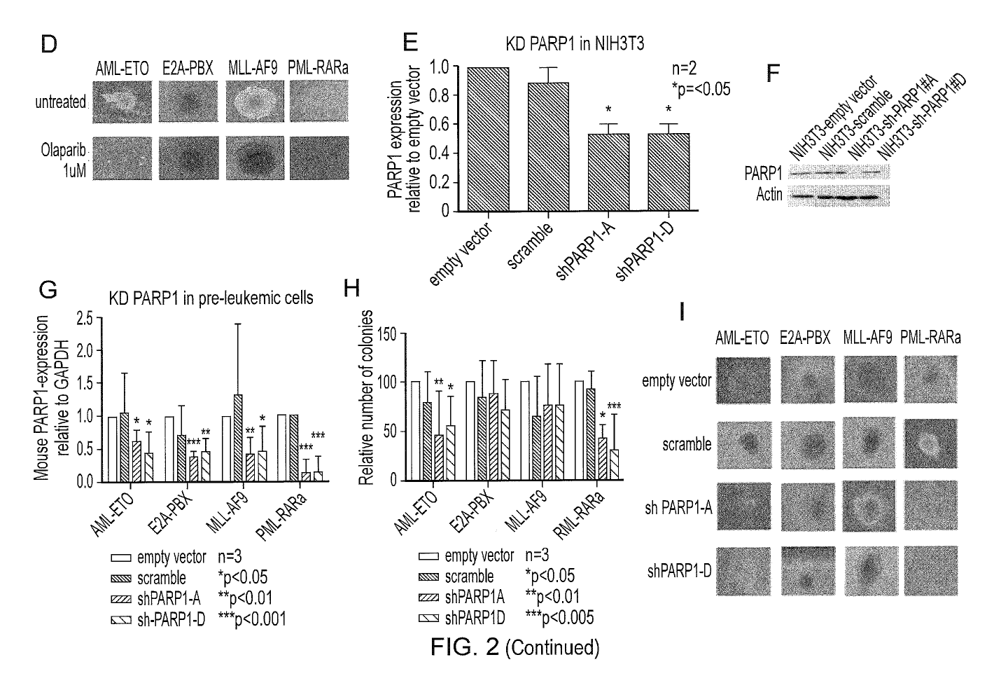

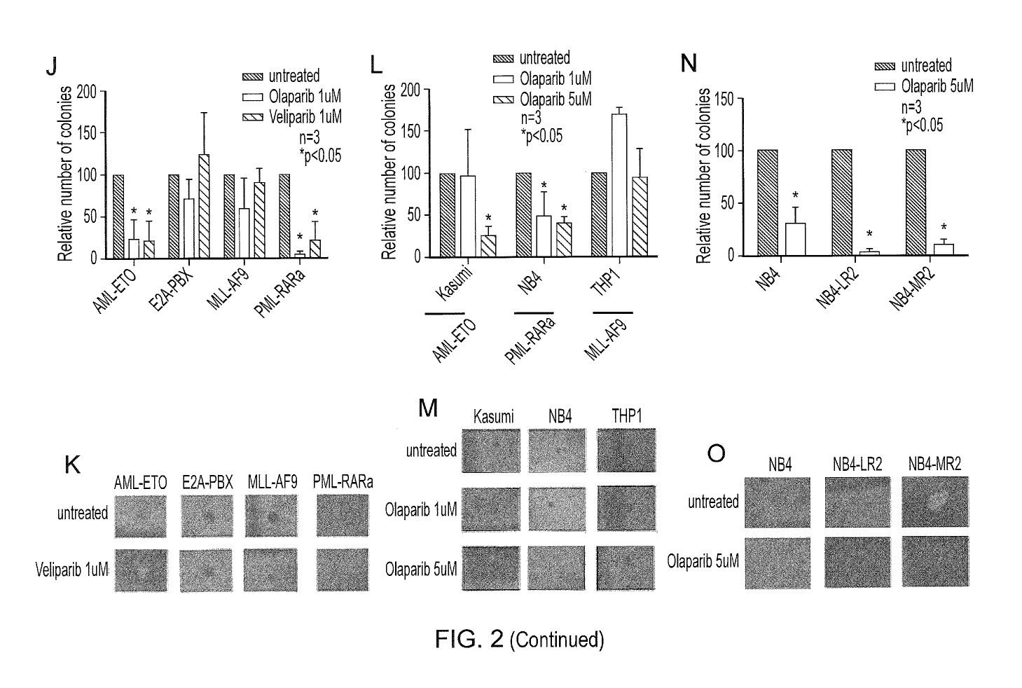

We investigated the effect of the PARP inhibitor Olaparib on clonogenic ability of primary murine leukemic cells carrying the most common leukaemia associated transcription factors (LATFs) including AML1-ETO, PML-RARα and MLL-AF9 (and E2A fusion as a control) generated by the retroviral transduction/transformation assay (RTTA), which has been extensively used in our and other labs to develop disease models and characterize the transformation mechanisms in acute leukaemia (FIG. 2A) (Kwok, Zeisig et al. 2006, Cheung, Chan et al. 2007, Zeisig, Kwok et al. 2007, Kwok, Zeisig et al. 2009, Zeisig and So 2009, Yeung, Esposito et al, 2010, Smith, Yeung et al. 2011). Briefly, c-kit positive progenitor cells of wild type Ly5.1 mouse bone marrow transduced with a retroviral vector expressing the LATFs of interest were plated into a methylcellulose medium supplemented with appropriate myeloid cytokines. Replating was performed every week to generate primary cell lines for further analysis. Non-transformed cells form colonies only in the initial plating and are lost during the serial replating (FIG. 2A). As previously done for bone marrow c-kit+ cells, we treated these primary leukaemic cells with escalating doses of Olaparib. As shown in FIG. 2B, AML-ETO and PML-RARα cells proved to be extremely sensitive to Olaparib with an IC50 of 33.4 nM and 15.8 nM, respectively. Further experiments were performed using Olaparib 1 uM. After exposure to Olaparib 1 uM primary transformed cells carrying AML1-ETO or PML-RARa displayed a significant reduction in colony forming ability (70-90% p<0.05) as compared with MLL-AF9 or E2A-PBX transformed cells (FIG. 2C-D). Additional replating of those colonies in semi-solid medium containing Olaparib further reduced the number of colony formed by AML1-ETO transformed cells (90% p<0.05) but not those by E2A-PBX and MLL-AF9 transformed cells (FIG. 2C-D). In order to confirm the specificity of the observed data we performed the same experiments with a different PARPi, Veliparib (ABT-888). Veliparib is an inhibitor of PARP1/2 produced by Abbott and widely used in clinical trials. Veliparib shows similar albeit less potency in trapping PARP1/2 on DNA damage sites. More importantly, Veliparib treatment reduced colonies of AML1-ETO and PML-RARα cells with no effect on E2A-PBX and MLL-AF9 (FIGS. 2J and K). No significant differences were found between Olaparib and Veliparib. In order to exclude any off target effects of these drugs and confirm that PARP1 targeting impairs the survival of AML1-ETO and PML-RARα cells, we genetically ablated PARP1 expression via shRNA, We cloned two shRNA against mouse PARP1 into retroviral vectors and tested their knockdown (KD) efficacy in NIH3T3 cell line. shPARP1-A and D were both able to reduce the expression of PARP1 as confirmed by qPCR (FIG. 2E) and western blot (FIG. 2F). We used these shRNA in a clonogenic assay as previously described for the drugs. shPARP1 dramatically affected the survival of AML1-ETO or PML-RARα colonies (FIG. 2 H-I). PARP1 Knock down efficiency in leukaemic cells was also confirmed by qPCR (FIG. 2G).

Although the RTTA provides us with a unique model to test the specific correlation between cytogenetics and PARPi treatment outcome, this assay does not recapitulate the complex genetic background of human patients, who accumulate multiple mutations in multiple genes. These mutations may have an important impact on the mechanisms of resistance to a particular treatment. By using three human cell lines, Kasumi, NB4 and THP1, established by leukaemic patients carrying respectively the oncofusion proteins AML1-ETO, PML-RARα and MLL-AF9, we could confirm the efficacy of Olaparib treatment in both Kasumi and NB4, in agreement with the data generated with mouse leukaemic cell lines (FIG. 2 L-M). More importantly, by employing two APL cell models known to be resistant to ATRA, NB4-LR2 (Roussel and Lanotte 2001) and NB4-MR2 (McNamara, Wang et al. 2008), we found these two cells lines been extremely sensitive to Olaparib (FIG. 2 N-O), indicating that Olaparib can be an attractive therapeutic option for ATRA resistant APL patients.

### PARPi Treatment Induces Apoptosis and Senescence in AML1-ETO and PML-RARα Leukaemic Cells.

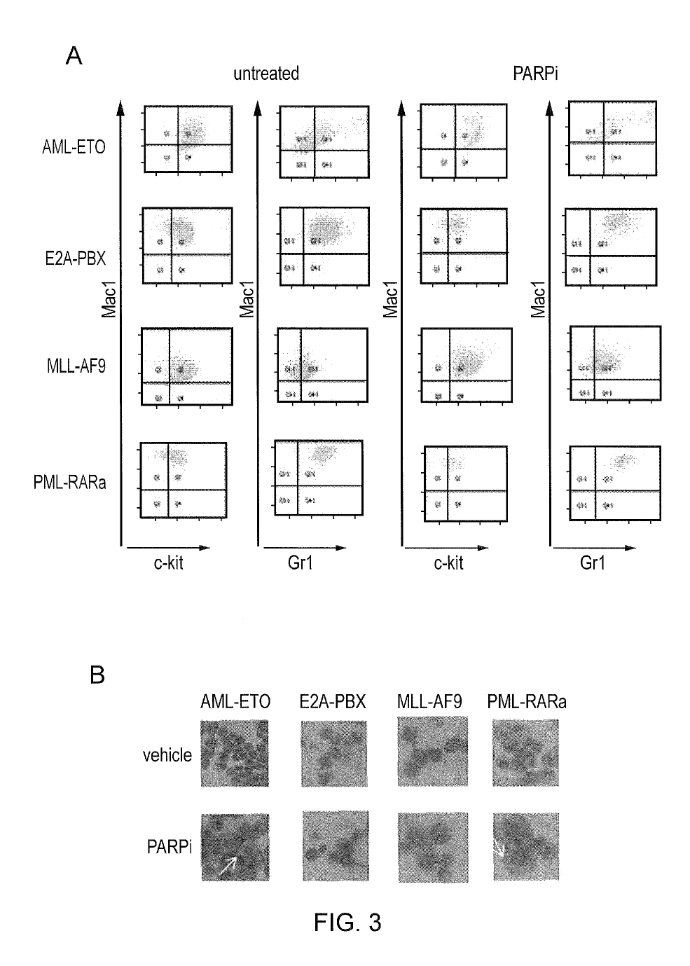

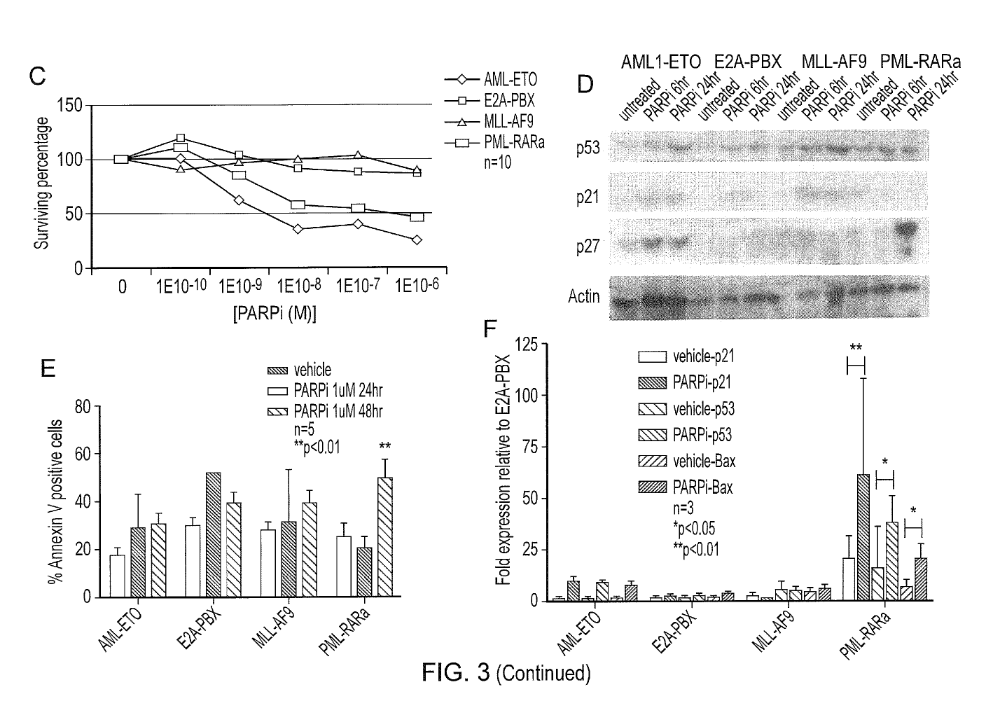

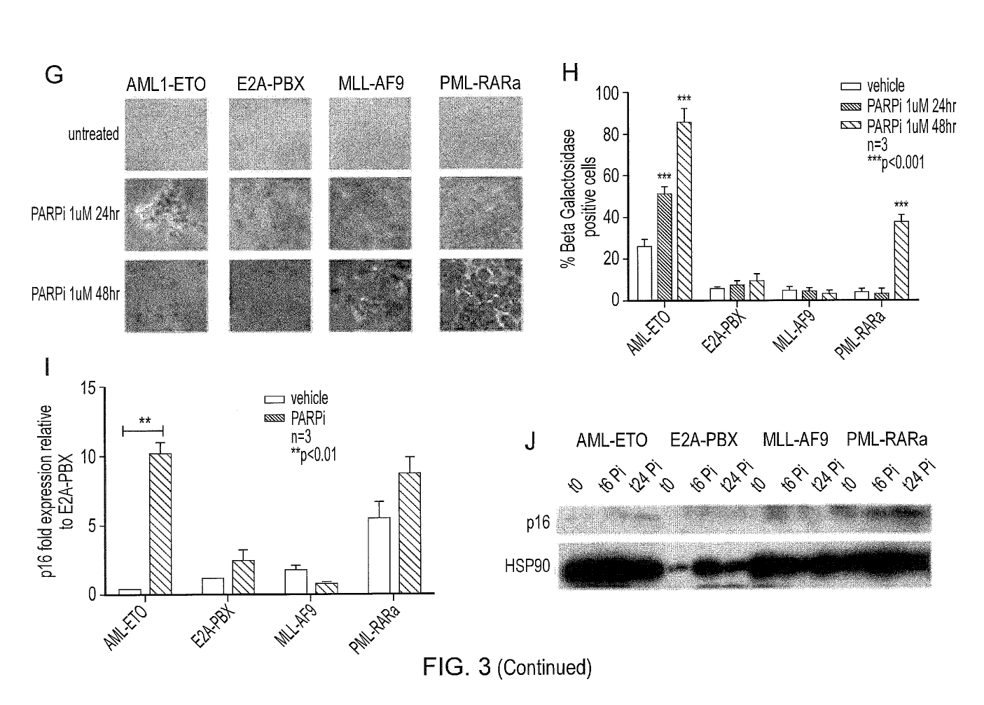

We reasoned that upon PARPi treatment, HR-proficient cells would transiently activate ATM-Chk2-p53 pathway, an early response in cells which undergo a genotoxic stress (Kastan and Bartek 2004), resulting in transient cell cycle arrest to allow DNA repair, whereas cells unable to repair the DNA damage would accumulate DNA damage, undergo differentiation, permanent cell cycle arrest (senescence) or apoptosis. Indeed, PARPi treatment induced differentiation in AML1-ETO or PML-RARα transformed cells, whereas no morphological or immunophenotypic difference was observed for E2A-PBX and MLL-AF9 transformed cells before and after exposure to PARPi (FIG. 3A-B). Consistent with this result and the results of clonogenic assay, PARPi treatment suppressed the proliferation, as determined by MTS assay (FIG. 3C), and induced cell cycle arrest in both AML-ETO and PML-RARα leukaemic cells, as shown by the accumulation of p27, inhibitor of Cyclin E and cyclin A dependent CDK2, by western blot (FIG. 3D). Further experiments indicated that PARPi enhanced apoptosis of AML1-ETO and PML-RARα transformed cells, as indicated by Annexin V staining (FIG. 3E). This effect is particularly pronounced in PML-RARα cells 48 hrs after treatment (FIG. 3E). Consistent with these results, high levels of p53 target genes, p21 and Bax mRNA were observed in AML1-ETO and PML-RARα leukaemic cells treated with PARPi for 48 hrs (FIG. 3F). Notably, PARPi treatment strongly induced senescence in AML1-ETO and, to a less extent, in PML-RARα cells, as confirmed by β-galactosidase staining (FIG. 3G-H) and p16 expression (FIG. 3I-J).

### AML1-ETO and PML-RARα Cells Show a Defect in HR Pathway.

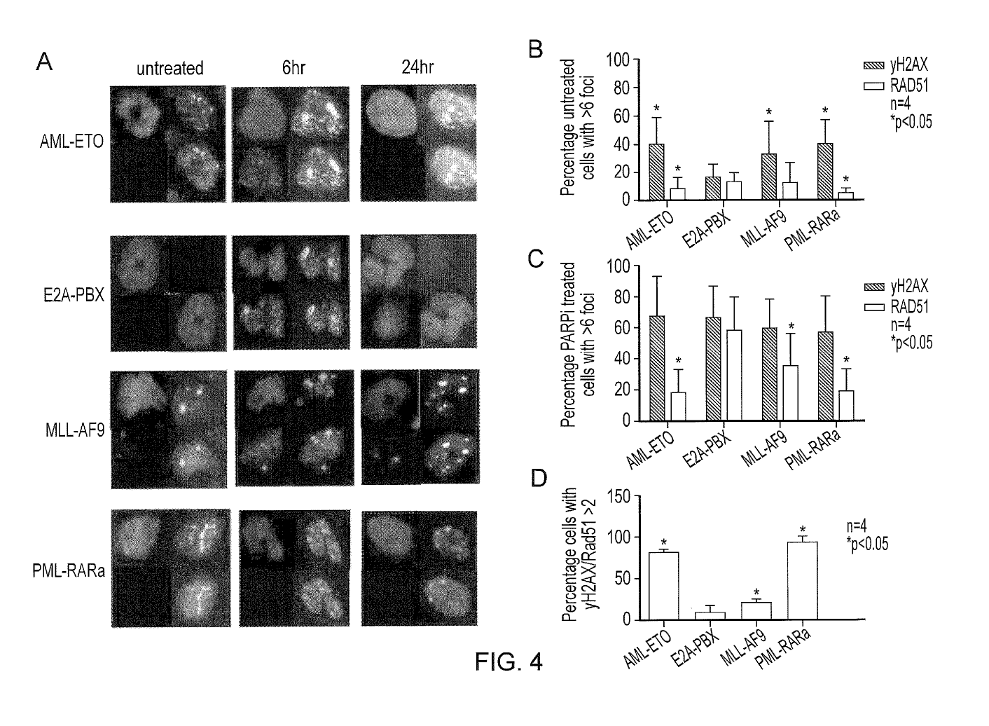

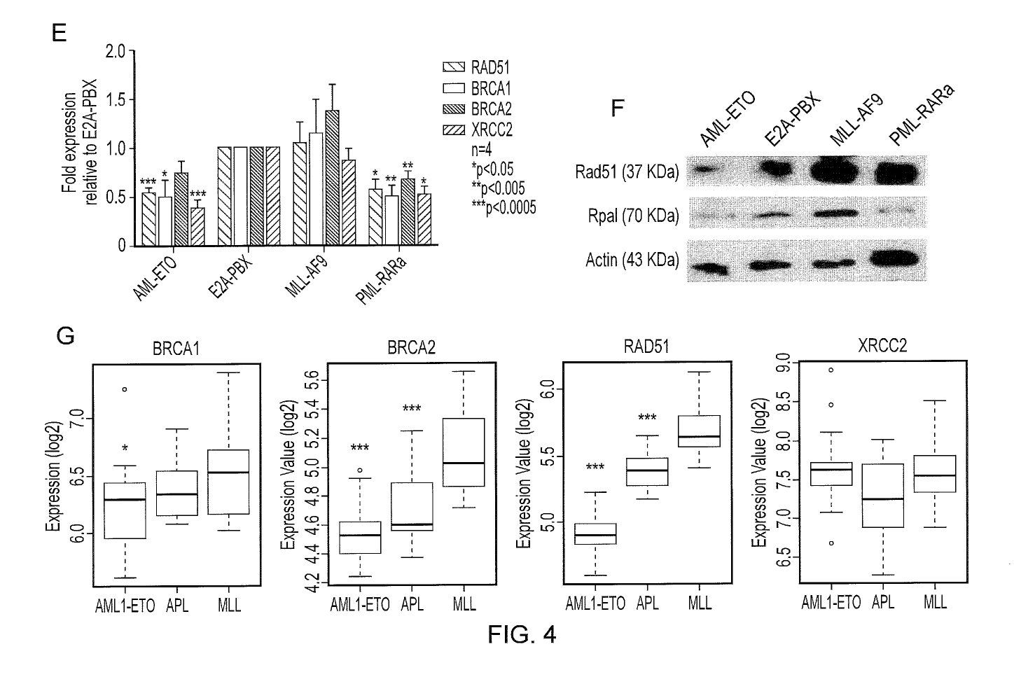

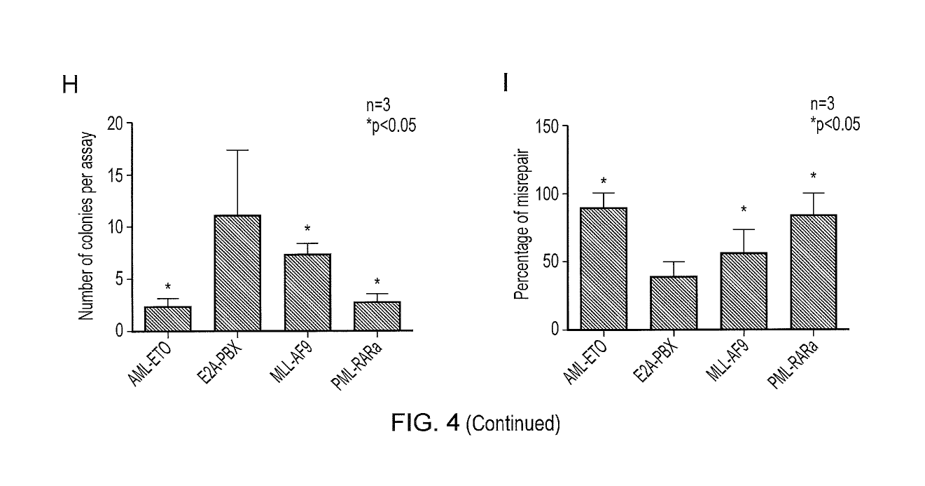

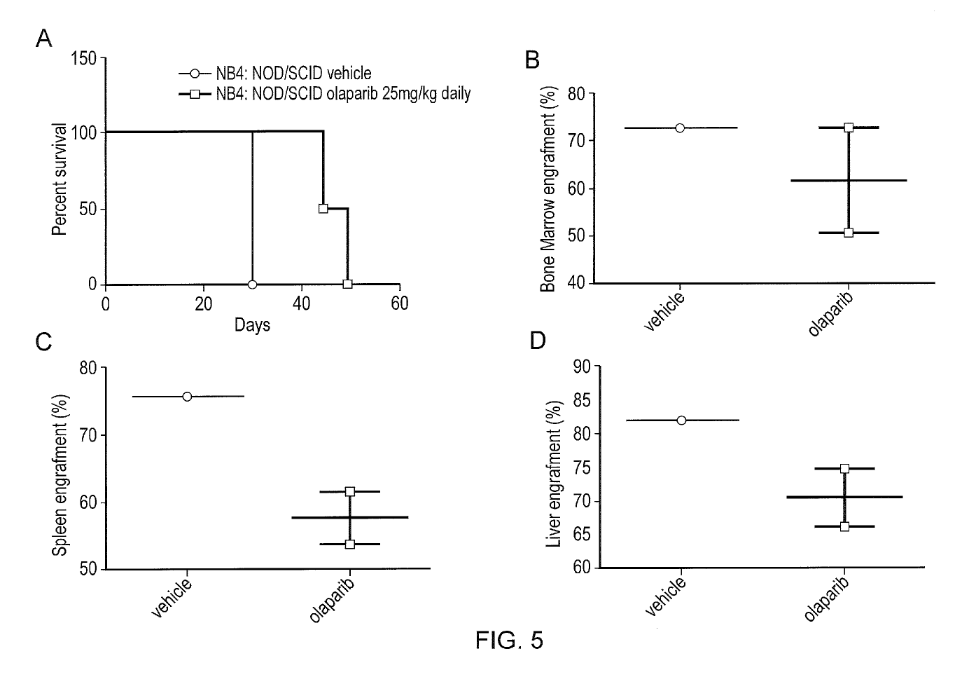

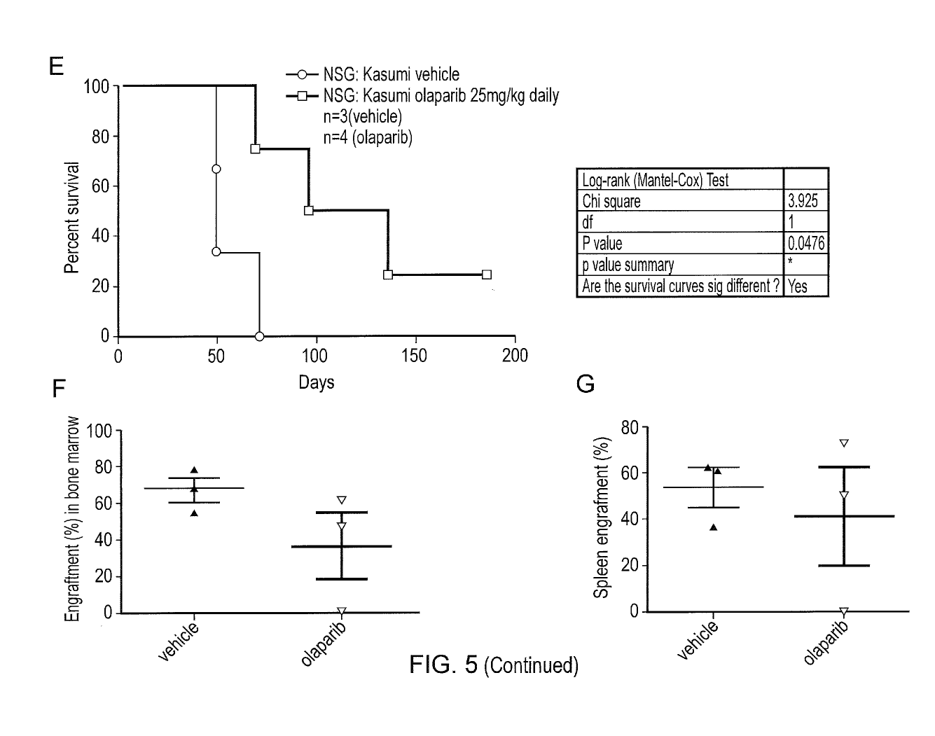

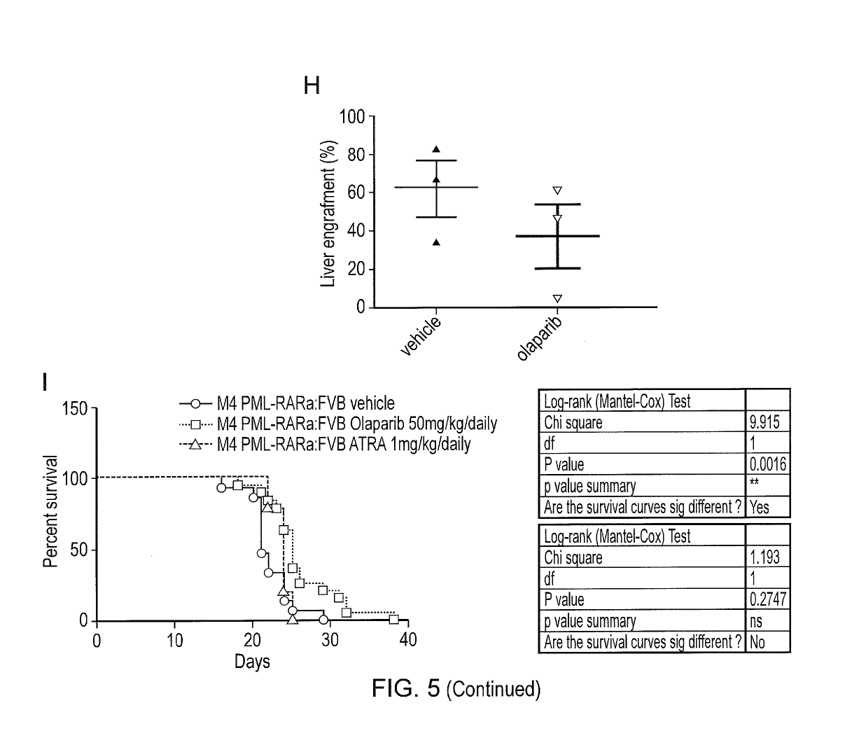

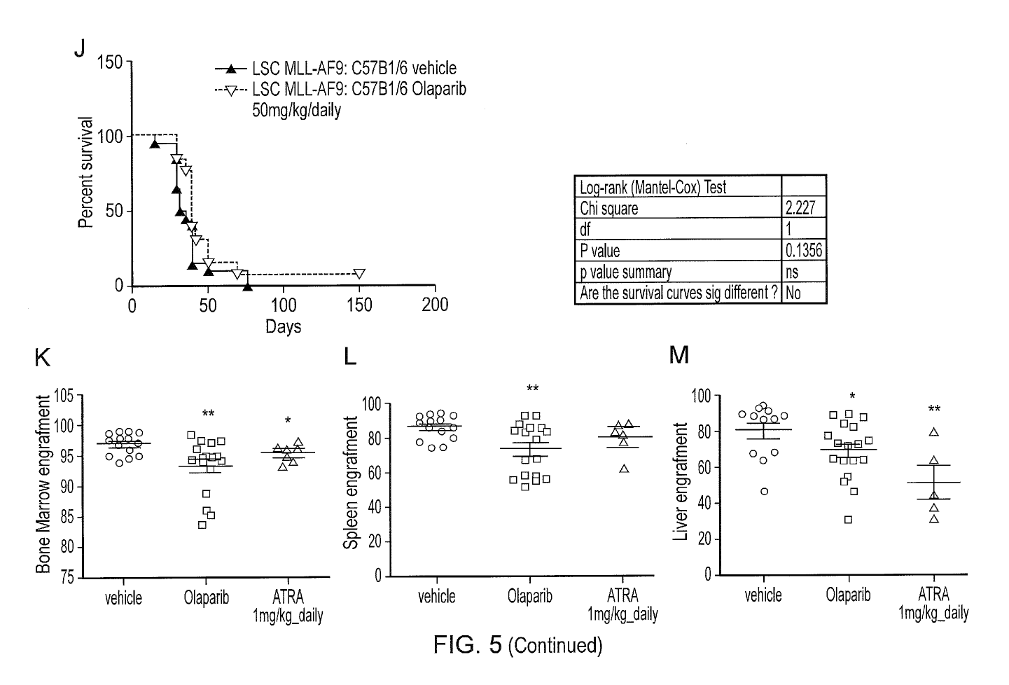

As the rationale behind the sensitivity to PARPi is a defect in DDR, we assessed the DNA damage and the kinetic of DDR of leukemic cells by analyzing the frequency of phosphorylated γ-H2AX foci by immunofluorescence assays. Ser-139 phosphorylation of H2AX, the minor histone H2A variant, is considered as an early cellular response to DSBs and the most well established chromatin modifications linked to DNA damage and repair. Indeed this event allows chromatin modifications, which increase DNA accessibility, leading to the recruitment and accumulation of specific DNA damage-response proteins at DNA ends. Untreated AML1-ETO, PML-RARα and MLL-AF9 transformed cells displayed significantly higher levels of yH2AX-positive DNA damage foci, indicative of DNA damage defect (FIG. 4A-B, black bars). Upon PARPi treatment for 6 hours, both PARPi resistant (E2A-PBX and MLL-AF9 cells) and sensitive cells (AML1-ETO and PML-RARα) showed a robust induction in y-H2AX-positive DNA damage foci (FIG. 4A-C black bars), suggesting that PARPi treatment induces DNA damage regardless the onco-fusion expressed by the cells.

As HR is the major pathway for repairing DSB in cycling cells and PARPi have been demonstrated to selectively target HR deficient cells (Bryant, Schultz et al. 2005, Farmer, McCabe et al. 2005), we investigated whether PARPi resistant cells were able to recruit Rad51 to DNA damage sites, as readout of HR efficiency. Upon PARPi treatment, E2A-PBX leukemic cells and, to a less extent MLL-AF9 cells, were able to form RAD51 foci (FIG. 4C white bars), whereas no significant Rad51 recruitment was observed in AML1-ETO and PML-RARα pre-leukemic cells (FIG. 4C white bars), confirming the hypothesis that AML1-ETO and PML-RARα cells are HR deficient, further corroborated by the ratio between yH2AX foci and Rad51 foci, which was more than 2 in 81% and 93% of AML1-ETO and PML-RARα, respectively (FIG. 4D). RT-qPCR analysis confirmed a decreased expression of Rad51, Brca1 and Xrcc2 in both AML-ETO and PML-RARα cells (FIG. 4E). Western blot analysis show reduced levels of Rad51 protein in AML1-ETO cells and of Rpa1 protein in both AML1-ETO and PML-RARα leukaemic cells (FIG. 4F). The different expression levels of BRCA1, BRCA2, RAD51 and XRCC2 among the AML subtypes was confirmed also in human patients by analysing an Affymetrix gene expression database (GEO accession: GSE1159) of leukaemic samples from AML1-ETO (22 samples), APL (18 samples), MLL (17 samples) (Valk, Verhaak et al. 2004) (FIG. 4G).

The synthetic lethality of PARPi has been more recently associated with enhancement of error-prone Non Homologus End Joining (NHEJ) which is responsible for genomic instability and eventual toxicity (Patel, Sarkaria et al. 2011). In order to assess the NHEJ status of our leukaemic cells we performed an in vivo plasmid end-joining assay (Baumann and West 1998). Briefly a Double Strand Break (DSB) is generated in the LacZ gene sequence of the plasmid PUC18 by EcoRI digestion. Nuclear extracts from leukaemic cells carrying the above mentioned onco-fusion proteins are then incubated with the digested PUC18 plasmid for 24 hours at 18° C., followed by DNA purification and transformation in Escherichia Coli. The results show that E2A-PBX and MLL-AF9 nuclear extracts produce on average a higher total number of colonies compared to AML-ETO and PML-RARα (FIG. 4H). Moreover, these latter nuclear extracts generated a higher frequency of misrepaired bacterial colonies, as assessed by the ratio between blue colonies (where efficient repair took place) and total colonies (FIG. 4I). Therefore these data indicate that AML-ETO and PML-RARα nuclear extracts have a reduced ability to repair DSBs and that this repair is accompanied with an increased error rate, confirming the hypothesis that AML1-ETO and PML-RARα leukaemic cells not only are HR deficient but are also prone to accumulate genomic instability.

PARPi Prolong the Survival of Mice Injected with AML1-ETO and PML-RARα Leukemic Cells.

Our data indicate the human cell lines Kasumi and NB4 are sensitive to PARPi treatment in vitro. In order to test the efficacy of this treatment in vivo, we transplanted Kasumi and NB4 cell lines into a small cohort of sublethally irradiated NOD/SCID/IL2Rg−/− (NSG) mice by intra-femoral and intravenous injection, respectively. In vivo intra-peritoneal injections with Olaparib 25 mg/kg were performed daily for two weeks. The maximum dose tolerated was calculated by in vivo dose-response experiments. Mice were monitored daily until they developed symptoms of leukaemia, when they were culled and bone marrow, spleen and liver harvested and analyzed by FACS. As indicated in FIG. 5 Olaparib treatment significantly delayed the onset of the leukaemia driven by PML-RARα (panel A) and AML1-ETO (panel E) oncogenes. The median survival of NB4:NSG mice increased from 30 to 47 days, whereas the median survival of Kasumi:NSG mice increased from 50 to 115 days, with a variable reduction in engraftment in bone marrow, spleen and liver. This experiment is still ongoing and the treated group still alive, therefore we expect an even more profound impact on survival.

More importantly our data demonstrate that ATRA resistant PML-RARα cells are sensitive to PARPi (FIG. 2J-K) indicating that PARPi may have a wider therapeutic window and potentially become the elective treatment for ATRA resistant leukaemic patients, a very important finding as no therapeutic option is currently available to these patients. To test the efficacy of this treatment in vivo, we used a well established ATRA resistant mouse model (Kogan, Hong et al. 2000), based on mouse leukemic cells carrying a mutated form of PML-RARα (M4) which cannot be bound by ATRA and that resemble the mutation found in human patients (Roussel and Lanotte 2001). Briefly we intravenously injected M4 PML-RARα cells in sub-lethally irradiated FVB mice. In vivo intra-peritoneal injection with Olaparib 50 mg/kg were performed daily for two weeks. The maximum dose tolerated was calculated by in vivo dose-response experiments. Mice were monitored daily until they developed symptoms of leukaemia, when they were culled and bone marrow, spleen and liver harvested and analyzed by FACS. As control we injected leukaemic MLL-AF9 into sub-lethally irradiated C57Bl/6 mice and distributed them in two groups, as previously described for M4-PML-RARα cells. Leukaemic MLL-AF9 were generated by injecting MLL-AF9 pre-leukaemic cells (pre-LSC), obtained by bone marrow from wild type Ly5.1 mouse through the RTTA assay, into irradiated mice expressing the Ly5.2 marker. The cells harvested from primary mice transplanted with pre-LSCs, were defined as leukaemic (LSCs). These cells phenocopy better than pre-LSCs primary human AML samples as it is well known that in vivo, epigenetic modifications and other mutations will cooperate with the oncofusion genes to further drive the leukemogenic process to the LSC stage. As shown in FIG. 5 Olaparib treatment significantly delayed the onset of the leukaemia driven by ATRA resistant M4-PML-RARα (Panel I, K, L and M), while it did not have any significant effect on mice developing MLL-AF9 driven leukaemia (panel J), indicating that Olaparib may represent a novel therapeutic option for ATRA resistant leukaemic patients. More experiments are currently ongoing to test the efficacy of Olaparib in combination with ATRA or standard chemotherapy (doxorubicin in combination with ara-C), in mouse models of APL and AML, respectively.

### Summary

These results show for the first time that PARPi selectively target leukaemic cells carrying the onco-fusion proteins AML1-ETO and PML-RARα while having a minimal toxicity to the normal counterparts, showing that selective use of PARPi can be highly beneficial to certain subgroups of AML patients.

Surprisingly, although MLL leukaemic cells show a certain level of DNA damage even in the untreated condition (FIG. 4A, and black bars in FIG. 4B-C), recruiting Rad51 to a less extent of E2A-PBX cells (FIG. 4A, and white bars in FIG. 4B-C), we found them to be resistant to PARPi suggesting potential back-up pathways, which may offset the effects associated with DDR and allow MLL-rearranged leukaemia to cope with genomic instability. These results highlight that although genomic instability is a common feature of leukaemia, not all AML patients may be suitable for PARPi treatment. Therefore cytogenetics data is extremely important to identify the right AML patients to treat with PARPi.

Genomic instability is a common feature of leukaemic cells. This phenomenon is associated with genetic mutations in specific players of DNA damage repair in some inherited leukaemias, such as the ones developed by Fanconi Anemia and Bloom's syndrome patients (Suhasini and Brosh 2012). In sporadic leukaemias, the mutations behind this genomic instability are unknown but increasing body of evidences indicate increased ROS generation by the activated tyrosine kinases BCR-ABL1 and FLT3-ITD (Sallmyr, Fan et al. 2008) and inhibition of DNA repair by onco-fusion proteins such as AML1-ETO and PML-RARα (Alcalay, Meani et al. 2003, Krejci, Wunderlich et al. 2008, Boichuk, Hu et al. 2011) may be the main driving forces. Therefore by characterizing a DNA repair defect in leukaemia driven by AML1-ETO and PML-RARα fusion proteins and by demonstrating for the first time their sensitivity to PARPi in both the mouse cells and more importantly in the human cells model, the present invention provides the use of PARPi as single agents or in combination with chemotherapy for the treatment of these specific subgroups of leukaemia.

Based on shared features between AML1-ETO and CBFβ-SMMHC, where AML1/CBFβ heterodimers regulate normal and malignant haematopoiesis, we envisage that PARPi treatment may be suitable also to patients expressing the onco-fusion protein CBFβ-SMMHC, resulting from the translocation t(16;16) or the inversion inv(16). Indeed both the two fusion proteins AML1-ETO and CBFβ-SMMHC have been proven to repress normal AML-1-CBFβ heterodimer dependent transcription of the same target genes and haematopoietic development (Speck and Gilliland 2002). This indicates that CBFβ-SMMHC may also affect a variety of genes involved in DDR, such as MPG, OGG1, POLD2, POLD3, POLE and ATM, as reported for AML1-ETO (Alcalay, Meani et al. 2003, Krejci, Wunderlich et al. 2008).

Moreover, by finding MLL-AF9 leukaemic cells been PARPi resistant, this study shows that not all AML patients may benefit from PARPi treatment and that identification of patients eligible to PARPi must rely on cytogenetics. PARPi are thus a novel targeted therapeutic alternative to chemotherapy with less toxicity and an improved anti tumour effect. Our data also indicate PARPi can provide a reliable therapeutic alternative to APL patients resistant to ATRA treatment. Furthermore, molecular dissection of the mechanisms underlying the PARPi resistance in the refractory subgroups will also facilitate the development of effective strategy to overcome the drug resistance.

Our data clearly indicate that AML1-ETO and PML-RARα leukaemic cells are uniquely sensitive to PARPi, thus providing the use of PARPi as single agents or in combination with chemotherapy for the treatment of AML1-ETO and PML-RARα leukaemias. Data generated by employing the human cell lines Kasumi and NB4 have also provided strong evidence that PARPi are efficacious in human cells.

### Further Studies

### 1) Molecular Mechanisms of PARPi Sensitivity

Although last generation PARPi have been significantly improved and have better efficacy and specificity, Olaparib, as well as other PARPi such as Veliparib (ABT-888) and rucaparib, inhibit PARP1 and PARP2 with similar potency (Wahlberg, Karlberg et al. 2012). Relative expression of PARP1 and PARP2 may account for redundancy and resistance of particular cell types. Therefore further studies were conducted studying PARP inhibition using shRNAs targeting PARP1 and PARP2. As predicted, only depletion of PARP1 recapitulates the phenotype observed in AML-ETO and PML-RARa leukaemic cells with Olaparib as previously shown (Bryant, Schultz et al. 2005, Farmer, McCabe et al. 2005) and the genetic approach has a less profound and toxic effect (Murai, Huang et al. 2012).

Our data indicate AML1-ETO and PML-RARα onco-fusion proteins induce DNA damage by impairing HR-DDR. While previous data have confirmed PML-RARα onco-fusion protein depresses the expression of Rad51 (Alcalay, Meani et al. 2003), this is the first report correlating AML-ETO onco-fusion protein to reduced levels of Rad51. ChIP analyses will be performed to determine if Rad51 is a direct target of AML-ETO, PML-RARα and CBFβ-SMMHC. Functional rescue will also be performed to assess the significance of DDR in the observed PARPi sensitivity by expressing the suppressed DDR components (e.g., Rad51) in PARPi sensitive cells.

### 2) Human Leukaemic Cells

Our in vivo data provide strong indications on the efficacy of PARPi in both the mouse (C57Bl/6, FVB mice) and humanized mouse models (NSG mice) of AML1-ETO and PML-RARα leukaemia.

Primary AML cells from patients will also be tested for PARPi sensitivity by means of proliferative assay, CFC assay, LTC-IC limiting dilution assay and by in vivo treatment in xenograft model as we previously described (Yeung, Esposito et al. 2010).

### REFERENCES

Alcalay, M., N. Meani, et al. (2003). “Acute myeloid leukemia fusion proteins deregulate genes involved in stem cell maintenance and DNA repair.” J Clin Invest 112(11): 1751-1761.
Baumann, P. and S. C. West (1998). “DNA end-joining catalyzed by human cell-free extracts.” Proc Natl Acad Sci USA 95(24): 14066-14070.
Boichuk, S., L. Hu, et al. (2011). “Functional connection between Rad51 and PML in homology-directed repair.” PLoS One 6(10): e25814.
Brightwell, M. and S. Shall (1971). “Poly(adenosine diphosphate ribose) polymerase in Physarum polycephalum nuclei.” Biochem J 125(3): 67P.
Bryant, H. E., N. Schultz, et al. (2005). “Specific killing of BRCA2-deficient tumours with inhibitors of poly(ADP-ribose) polymerase.” Nature 434(7035): 913-917.
Cheung, N., L. C. Chan, et al. (2007). “Protein arginine-methyltransferase-dependent oncogenesis.” Nat Cell Biol 9(10): 1208-1215.
Farmer, H., N. McCabe, et al. (2005). “Targeting the DNA repair defect in BRCA mutant cells as a therapeutic strategy.” Nature 434(7035): 917-921.
Fong, P. C., D. S. Boss, et al. (2009). “Inhibition of poly(ADP-ribose) polymerase in tumors from BRCA mutation carriers.” N Engl J Med 361(2): 123-134.
Gaymes, T. J., S. Shall, et al. (2009). “Inhibitors of poly ADP-ribose polymerase (PARP) induce apoptosis of myeloid leukemic cells: potential for therapy of myeloid leukemia and myelodysplastic syndromes.” Haematologica 94(5): 638-646.
Grimwade, D., P. Vyas, et al. (2010). “Assessment of minimal residual disease in acute myeloid leukemia.” Curr Opin Oncol 22(6): 656-663.
Helleday, T. (2011). “The underlying mechanism for the PARP and BRCA synthetic lethality: clearing up the misunderstandings.” Mol Oncol 5(4): 387-393.
Kastan, M. B. and J. Bartek (2004). “Cell-cycle checkpoints and cancer.” Nature 432(7015): 316-323.
Krejci, O., M. Wunderlich, et al. (2008). “p53 signaling in response to increased DNA damage sensitizes AML1-ETO cells to stress-induced death.” Blood 111(4): 2190-2199.
Krishnakumar, R. and W. L. Kraus (2010). “The PARP side of the nucleus: molecular actions, physiological outcomes, and clinical targets.” Mol Cell 39(1): 8-24.
Kwok, C., B. B. Zeisig, et al. (2006). “Forced homo-oligomerization of RARalpha leads to transformation of primary hematopoietic cells.” Cancer Cell 9(2): 95-108.
Kwok, C., B. B. Zeisig, et al. (2010). “The role of CBFbeta in AML1-ETO's activity.” Blood 115(15): 3176-3177.
Kwok, C., B. B. Zeisig, et al. (2009). “Transforming activity of AML1-ETO is independent of CBFbeta and ETO interaction but requires formation of homo-oligomeric complexes.” Proc Natl Acad Sci USA 106(8): 2853-2858.
Patel, A. G., J. N. Sarkaria, et al. (2011). “Nonhomologous end joining drives poly(ADP-ribose) polymerase (PARP) inhibitor lethality in homologous recombination-deficient cells.” Proc Natl Acad Sci USA 108(8): 3406-3411.
Rouleau, M., A. Patel, et al. (2010). “PARP inhibition: PARP1 and beyond.” Nat Rev Cancer 10(4): 293-301.
Sallmyr, A., A. Miller, et al. (2004). “Expression of DNA-dependent protein kinase in human granulocytes.” Cell Res 14(4): 331-340.
Smith, L. L., J. Yeung, et al. (2011). “Functional crosstalk between Bmi1 and MLL/Hoxa9 axis in establishment of normal hematopoietic and leukemic stem cells.” Cell Stem Cell 8(6): 649-662.
So, C. W. (2009). “An overview: from discovery of candidate mutations to disease modeling and transformation mechanisms of acute leukemia.” Methods Mol Biol 538: 1-5.
Suhasini, A. N. and R. M. Brosh, Jr. (2012). “Fanconi anemia and Bloom's syndrome crosstalk through FANCJ-BLM helicase interaction.” Trends Genet 28(1): 7-13.
Takacova, S., R. Slany, et al. (2012). “DNA damage response and inflammatory signaling limit the MLL-ENL-induced leukemogenesis in vivo.” Cancer Cell 21(4): 517-531.
Turner, N., A. Tutt, et al. (2004). “Hallmarks of ‘BRCAness’ in sporadic cancers.” Nat Rev Cancer 4(10): 814-819.
Tutt, A., M. Robson, et al. (2010). “Oral poly(ADP-ribose) polymerase inhibitor olaparib in patients with BRCA1 or BRCA2 mutations and advanced breast cancer: a proof-of-concept trial.” Lancet 376(9737): 235-244.
Viale, A., F. De Franco, et al. (2009). “Cell-cycle restriction limits DNA damage and maintains self-renewal of leukaemia stem cells.” Nature 457(7225): 51-56.
Wahlberg, E., T. Karlberg, et al. (2012). “Family-wide chemical profiling and structural analysis of PARP and tankyrase inhibitors.” Nat Biotechnol 30(3): 283-288.
Weston, V. J., C. E. Oldreive, et al. (2010). “The PARP inhibitor olaparib induces significant killing of ATM-deficient lymphoid tumor cells in vitro and in vivo.” Blood 116(22): 4578-4587.
Yan, M., E. Kanbe, et al. (2006). “A previously unidentified alternatively spliced isoform of t(8;21) transcript promotes leukemogenesis.” Nat Med 12(8): 945-949.
Yeung, J., M. T. Esposito, et al. (2010). “beta-Catenin mediates the establishment and drug resistance of MLL leukemic stem cells.” Cancer Cell 18(6): 606-618.
Yeung, P. L., N. G. Denissova, et al. (2011). “Promyelocytic leukemia nuclear bodies support a late step in DNA double-strand break repair by homologous recombination.” J Cell Biochem.
Zeisig, B. B., C. Kwok, et al. (2007). “Recruitment of RXR by homotetrameric RARalpha fusion proteins is essential for transformation.” Cancer Cell 12(1): 36-51.
Zeisig, B. B. and C. W. So (2009). “Retroviral/Lentiviral transduction and transformation assay.” Methods Mol Biol 538: 207-229.

All publications mentioned in the above specification are herein incorporated by reference. Various modifications and variations of the described embodiments of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method for predicting responsiveness of a subject to a poly-(ADP-ribose)-polymerase (PARP) inhibitor for treating acute myeloid leukaemia (AML), the method comprising determining whether a chromosomal abnormality selected from t(8;21), t(15;17), t(16;16) and inv(16) is present in a sample obtained from the subject; wherein the presence of the chromosomal abnormality is indicative of responsiveness of the subject to the PARP inhibitor for treating AML.

2. A method according to claim 1, wherein the PARP inhibitor is selected from olaparib, veliparib, CEP-8983 or a prodrug thereof, rucaparib, E7016, BMN-673, and INO-1001, and analogues and derivatives thereof.

3. A method according to claim 1 or claim 2, wherein the chromosomal abnormality is the translocation t(8;21) which results in expression of a fusion protein comprising acute myeloid leukemia-1 transcription factor and eight-twenty-one corepressor (AML1-ETO).

4. A method according to claim 1 or claim 2, wherein the chromosomal abnormality is the translocation t(15;17) which results in expression of a fusion protein comprising promyelocytic leukemia protein and retinoic acid receptor alpha (PML-RARα).

5. A method according to claim 1 or claim 2, wherein the chromosomal abnormality is the translocation t(6;16) which results in expression of a fusion protein comprising core binding factor β and smooth muscle myosin heavy chain (CBFβ-SMMHC).

6. A method according to claim 1 or claim 2, wherein the chromosomal abnormality is the inversion inv(16) which results in expression of a fusion protein comprising core binding factor β and smooth muscle myosin heavy chain (CBFβ-SMMHC).

7. A method according to any preceding claim, wherein the sample is derived from bone marrow or blood.

8. A method for treating a subject for acute myeloid leukaemia (AML), the method comprising:
(i) predicting responsiveness of the subject to a poly-(ADP-ribose)-polymerase (PARP) inhibitor by a method as defined in any preceding claim; and
(ii) treating the subject with a PARP inhibitor if the subject is predicted to be responsive thereto.

9. A method according to claim 8, wherein the PARP inhibitor is olaparib or veliparib.

10. A method according to claim 8 or claim 9, wherein the subject has the chromosomal translocation t(8;21) or t(16;16), or the chromosomal inversion inv(16).

11. A method according to claim 10, wherein the PARP inhibitor is administered to the subject in combination with a chemotherapeutic agent selected from cytarabine (ara-C) and/or an anthracycline.

12. A method according to claim 10, wherein the subject previously failed to respond to a chemotherapeutic agent selected from cytarabine (ara-C) and/or an anthracyline.

13. A method according to claim 8 or claim 9, wherein the subject has the chromosomal translocation t(15;17).

14. A method according to claim 13, wherein the PARP inhibitor is administered to the subject in combination with all-trans-retinoic acid (ATRA) and/or an anthracycline.

15. A method according to claim 13, wherein the subject previously failed to respond to treatment with all-trans-retinoic acid (ATRA) and/or an anthracycline.

16. A method according to any of claims 11 to 15, wherein the anthracycline is daunorubicin or doxorubicin.

17. A method according to any of claims 8 to 16, wherein the subject is suffering from relapsed AML.

18. A method according to any of claims 8 to 17, wherein the subject is unsuitable for a hematopoietic stem cell transplant.

19. A poly-(ADP-ribose)-polymerase (PARP) inhibitor for use in treating acute myeloid leukaemia (AML) in a subject, wherein the subject has a chromosomal abnormality selected from t(8;21), t(15;17), t(16;16) and inv(16).

20. A PARP inhibitor for use according to claim 19, wherein the PARP inhibitor is olaparib.

21. A PARP inhibitor for use according to claim 19 or claim 20, wherein the subject has the chromosomal translocation t(8;21) or t(16;16), or the chromosomal inversion inv(16).

22. A PARP inhibitor for use according to claim 21, wherein the subject is resistant to treatment with cytarabine (ara-C) and/or an anthracyline.

23. A PARP inhibitor for use according to claim 19 or claim 20, wherein the subject has the chromosomal translocation t(15;17).

24. A PARP inhibitor for use according to claim 23, wherein the subject is resistant to treatment with all-trans-retinoic acid (ATRA) and/or an anthracycline.

25. A PARP inhibitor according to any of claims 19 to 24, for use in treating relapsed AML in a subject.

26. A PARP inhibitor for use according to any of claims 19 to 25, wherein the subject is unsuitable for a hematopoietic stem cell transplant.

27. A pharmaceutical combination comprising (i) a poly-(ADP-ribose)-polymerase (PARP) inhibitor and (ii) a chemotherapeutic agent and/or all-trans-retinoic acid (ATRA); for simultaneous, separate or sequential use in treating acute myeloid leukaemia (AML) in a subject.

28. A pharmaceutical combination for use according to claim 27, wherein the subject has a chromosomal abnormality selected from t(8;21), t(15;17), t(16;16) and inv(16).

29. A pharmaceutical combination for use according to claim 27 or claim 28, wherein the PARP inhibitor is selected from olaparib, veliparib, CEP-8983 or a prodrug thereof, rucaparib, E7016, BMN-673, and INO-1001, and analogues and derivatives thereof.

30. A pharmaceutical combination for use according to claim 29, wherein the PARP inhibitor is olaparib or veliparib.

31. A pharmaceutical combination for use according to any of claims 27 to 30, wherein the chemotherapeutic agent is selected from cytarabine (ara-C) and/or an anthracycline.

32. A pharmaceutical combination for use according to claim 31, wherein the anthracycline is daunorubicin or doxorubicin.
